(19) United States
(12) Patent Application Publication
(10) Pub. No.: US 2007/0021501 A1
(43) Pub. Date: Jan. 25, 2007

(54) Methods of treating epileptogenesis

(76) Inventors: Roy E. Twyman, Doylestown, PA (US); Boyu Zhao, Lansdale, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/481,626

(22) Filed: Jul. 6, 2006

Related U.S. Application Data
(60) Provisional application No. 60/698,625, filed on Jul. 12, 2005.

(51) Int. Cl. A61K 31/325

(52) U.S. Cl. 514/483; 514/489

(57) ABSTRACT

This invention is directed to methods for preventing, treating, reversing, inhibiting, or arresting epileptogenesis in a subject comprising administering to the subject in need thereof a therapeutically effective amount of a compound selected from the group consisting of Formula (I) and Formula (II), or a pharmaceutically acceptable salt or ester thereof,: 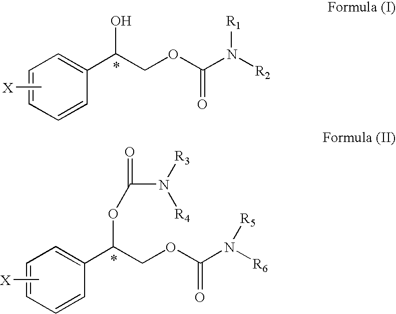
wherein phenyl is substituted at X with one to five halogen atoms selected from the group consisting of fluorine, chlorine, bromine and iodine; and, R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

### BACKGROUND OF THE INVENTION

1. Field of the Invention

This invention relates generally to the fields of pharmacology, neurology and psychiatry. In particular, the present invention provides methods for treating, preventing, reversing, arresting or inhibiting the development and maturation of seizures or seizure-related disorders. More specifically, this invention provides methods for the use of certain carbamate compounds to therapeutically or prophylactically treat, prevent, reverse, arrest or inhibit epileptogenesis.

2. Description of the Related Art

Injuries or trauma of various kinds to the central nervous system (CNS) or the peripheral nervous system (PNS) can produce profound and long-lasting neurological and psychiatric symptoms and disorders. One common mechanism for the production of these effects is the induction of seizure activity or seizure-like phenomena in the CNS or in the nerves and ganglia of the PNS. Symptomatic of paroxysmal disturbances in CNS or PNS electrical activity, seizures or seizure-like neurological mechanisms are believed to underlie many of the pathological phenomena in a wide variety of neurological and psychiatric disorders.

One serious neurological condition characterized by seizures is epilepsy. Epilepsy is a common but devastating disorder affecting more than two and a half million people in the United States alone. The term epilepsy refers to a disorder of brain function characterized by the periodic and unpredictable occurrence of seizures (See, The Treatment of Epilepsy, Principles & Practice, Third Edition, Elaine Wyllie, M.D. Editor, Lippincott Williams & Wilkins, 2001; Goodman & Gilman's The Pharmacological Basis of Therapeutics, 9^{th} edition, 1996). Seizures that occur without evident provocation are classified as epileptic. A subject is typically considered to suffer from epilepsy upon experiencing two or more seizures that occur more than 24 hours apart.

Clinically, an epileptic seizure results from a sudden and abnormal electrical discharge originating from a collection of interconnected neurons in the brain or elsewhere in the nervous system. Depending on the type of epilepsy involved, the resulting nerve cell activity may be manifested by a wide variety of clinical symptoms such as uncontrollable motor movements, changes in the patient's level of consciousness and the like.

On the basis of clinical and encephalographic phenomenon, four subdivisions of epilepsy are recognized: grand mal epilepsy (with subgroups: generalized, focal, jacksonian), petit mal epilepsy, psychomotor or temporal lobe epilepsy (with subgroups: psychomotor proper or tonic with adversive or torsion movements or masticatory phenomenon, automatic with amnesia, or sensory with hallucinations or dream states) and autonomic or diencephalic epilepsy (with flushing, pallor, tachycardia, hypertension, perspiration or other visceral symptoms).

While epilepsy is one of the foremost examples of a seizure-related disorder, a wide variety of neurological and psychiatric symptoms and disorders may have, as their etiology, seizures or related seizure-like neurological phenomenon. In simple terms, a seizure or a related seizure-like neurological phenomenon is a single discrete clinical event caused by an excessive electrical discharge from a collection of neurons or a seizure susceptible group of neurons through a process termed “ictogenesis.” As such, ictogenic seizures may be merely the symptom of a disease. However, epilepsy and other analogous seizure-related disorders are dynamic and often progressive diseases, with a maturation process characterized by a complex and poorly understood sequence of pathological transformations.

The development and maturation of such changes is the process of “epileptogenesis,” whereby the larger collection of neurons that is the normal brain is altered and subsequently becomes susceptible to abnormal, spontaneous, sudden, recurrent, excessive electrical discharges, i.e., seizures. The maturation of the epileptogenic process results in the development of an “epileptogenic focus,” whereby the collections of abnormally discharging neurons or neurons susceptible to seizures form localized groups or “epileptogenic zones” interspersed throughout the cortical tissue. The epileptogenic zones are biochemically inter-connected such that an abnormal ictogenic discharge is able to cascade from zone to zone. As epileptogenesis progresses, the involved areas of the nervous system become more susceptible to a seizure and it becomes easier for a seizure to be triggered, resulting in progressively debilitating symptoms of the seizure or seizure-related disorder.

While ictogenesis and epileptogenesis may have a common origin in certain biochemical phenomenon and common neuronal pathways in various diseases, the two processes are not identical. Ictogenesis is the initiation and propagation of a seizure in a discrete time and space, a rapid and definitive electrical/chemical event that occurs over a period of time ranging from seconds to minutes.

Comparatively, epileptogenesis is a gradual biochemical or neuronal restructuring process whereby the normal brain is transformed by ictogenic events into an epileptogenically focused brain, having neuronal circuitry that becomes sensitized and responsive to ictogenic events, making an individual increasingly susceptible to the recurrence of spontaneous, episodic, time-limited seizures, resulting in progressively debilitating symptoms of the seizure or seizure-related disorder and progressive non-responsiveness to treatment. The maturation of an “epileptogenic focus” is a slow biochemical and/or structural process that generally occur over months to years.

Epileptogenesis: A Two Phase Process:

“Phase 1 epileptogenesis” is the initiation of the epileptogenic process prior to the first epileptic seizure or seizure of an analogous seizure-related disorder, and is often the result of some kind of injury or trauma to the brain, i.e., stroke, disease (e.g., infection such as meningitis), or trauma, such as an accidental blow to the head or a surgical procedure performed on the brain.

“Phase 2 epileptogenesis” refers to the process during which brain tissue that is already susceptible to epileptic seizures or seizures of an analogous seizure-related disorder, becomes still more susceptible to seizures of increasing frequency and/or severity and/or becomes less responsive to treatment.

While the processes involved in epileptogenesis have not been definitively identified, some researchers believe that the up regulation of excitatory coupling between neurons, mediated by N-methyl-D-aspartate (NMDA) receptors, is involved. Other researchers implicate down regulation of inhibitory coupling between neurons, mediated by gamma-amino-butyric acid (GABA) receptors. Many other factors may be involved in this process relating to the presence, concentration or activity of NO (nitric oxide) or iron, calcium or zinc ions.

Although epileptic seizures are rarely fatal, large numbers of patients require medication to avoid the disruptive, and potentially dangerous consequences of seizures. In many cases, medication used to manage the epileptic seizures or seizures of an analogous seizure-related disorder is required for extended periods of time, and in some cases, a patient must continue to take such prescription drugs for life. Furthermore, such drugs can only be used for the management of symptoms and have side effects associated with chronic, prolonged usage.

A wide variety of drugs available for the management of epileptic seizures include older agents such as phenytoin, valproate and carbamazepine (ion channel blockers), as well as newer agents such as felbamate, gabapentin, topiramate and tiagabine. In addition, β-alanine has been reported to have anti-seizure activity, NMDA inhibitory activity and GABAergic stimulatory activity, but has not been employed clinically to treat epilepsy. Accepted drugs for the treatment of epilepsy are anticonvulsant agents or, more properly termed, anti-epileptic drugs (AEDs), wherein the term “anti-epileptic” is synonymous with “anti-seizure” or “anti-ictogenic”. These drugs therapeutically suppress seizures by blocking the initiation of a single ictogenic event. But it is believed that they are not prophylactically or therapeutically effective in influencing epileptogenesis.

In treating seizures for analogous seizure-related disorders, that is for diseases and disorders with seizure-like neurological phenomenon that may apparently be related to seizures disorders, such as mood cycling in Bipolar Disorder, impulsive behavior in patients with Impulse Control Disorders or for seizures resulting from brain injury, some AEDs may also be therapeutically useful. However, they are likewise unable to prophylactically or therapeutically prevent the initial development or progressive maturation of epileptogenesis to an epileptogenic focus that also characterizes analogous seizure-related disorders.

The poorly understood pathological mechanisms that underlie epileptogenesis certainly play a role in the development of epilepsy and analogous seizure-related disorders under a variety of clinical circumstances including spontaneous development or as a result of injury or trauma of many kinds to the central or peripheral nervous system.

Current epilepsy treatment is focused on suppressing seizure activity by administering AEDs after overt clinical epilepsy has developed. Although AEDs have positive effects in suppressing seizures, they have been universally unsuccessful in preventing epileptogenesis, i.e., the development or progression of epilepsy and other related seizure-like diseases. Even pretreatment with AEDs does not prevent the development of epilepsy after injury or trauma to the nervous system. Moreover, if therapy with AEDs is discontinued, the seizures typically recur and, in unfortunate instances, worsen with time. Currently, there is no known effective method for treating, preventing, reversing, arresting or inhibiting the onset and/or progression of epilepsy.

In addition, it is also believed that similar neurological mechanisms corresponding to epileptogenesis may be involved in the evolution and development of many seizure-related disorders clinically analogous to epilepsy that do not appear to be overtly “epileptic,” such as the initial development and progressive worsening observed in the mature disease state in Bipolar Disorder, Impulse Control Disorders, Obsessive-Compulsive disorders, Schizoaffective disorders and other psychiatric disorders.

Thus, despite the numerous drugs available for the treatment of epilepsy (i.e., through suppression of ictus epilepticus, i.e., the convulsions associated with epileptic seizures) and other analogous seizure-related disorders, there are no generally accepted drugs for treating, preventing, reversing, arresting or inhibiting the underlying process of epileptogenesis that may be etiologic in many devastating neurological and psychiatric disorders such as epilepsy and analogous seizure-related disorders.

Currently, there are no known methods of inhibiting the epileptogenic process to prevent the development of epilepsy or other analogous seizure-related disorders in patients who have not yet clinically shown symptoms thereof, but who unknowingly have the disease or are at risk of developing the disease. In addition, there are no known methods to prevent the development of or reverse the process of epileptogenesis, thus converting the collections of neurons in an epileptogenic zone which have been the source of or are susceptible or are capable of participating in seizure activity into nerve tissue that does not exhibit abnormal, spontaneous, sudden, recurrent or excessive electrical discharges or is not susceptible to or capable of such seizure activity. Furthermore, there are no approved or unapproved medications recognized as having such anti-epileptogenic properties, i.e., truly anti-epileptogenic drugs (AEGDS) (See, Schmidt, D. and Rogawski, M. A., Epilepsy Research, 2002, 50; 71-78).

Thus, there is a great need to develop safe and effective drugs and methods of treatment that effectively treat, prevent, arrest, inhibit and reverse epileptogenesis in seizure-related neurological and/or psychiatric disorders.

### SUMMARY OF THE INVENTION

This invention relates, in part, to methods and compounds useful for the treatment and/or prevention, arrest, inhibition and reversal of epileptogenesis in a patient who may but does not need to have the symptoms of epilepsy and/or an analogous seizure-related disorder.

This invention is based, in part, on the unexpected discovery that certain carbamate compounds, which are effective AEDs and can suppress epileptic seizures are, in addition, powerfully anti-epileptogenic and can prevent the occurrence of the development and maturation of the pathological changes in the nervous system that allow seizures and related phenomena to develop and/or spread and can reverse those changes. Thus, the carbamate compounds of the present invention, as used in the methods of this invention, are true AEGDs and have properties not possessed by any presently available medication or AED.

Therefore, in one aspect, the invention provides a method for treating and preventing seizures and seizure-related disorders in a subject in need thereof. In another aspect, the invention provides a method for arresting, inhibiting and reversing epileptogenesis in the subject. The method includes the step of prophylactically or therapeutically administering to the subject in need thereof an effective amount of a carbamate compound that treats, prevents, arrests, inhibits and reverses epileptogenesis in the subject.

In various embodiments, the invention provides methods of treating, preventing, reversing, arresting or inhibiting epileptogenesis. In certain embodiments, these methods comprise administering a prophylactically or therapeutically effective amount of a carbamate compound to the subject.

Accordingly, the present invention provides methods for treating, preventing, arresting, inhibiting and reversing epileptogenesis in a subject in need thereof comprising administering to the subject a prophylactically or therapeutically effective amount of a composition that comprises at least one compound of Formula 1 or Formula 2: 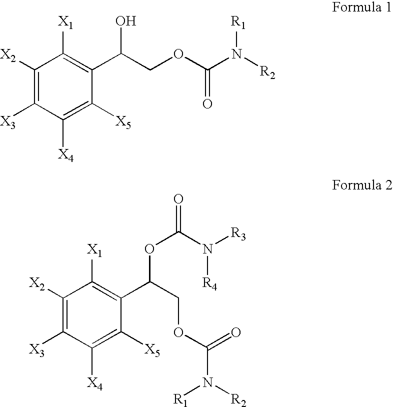
or a pharmaceutically acceptable salt or ester form thereof, wherein R₁, R₂, R₃ and R₄ are independently hydrogen or C₁-C₄ alkyl, wherein C₁-C₄alkyl is substituted or unsubstituted with phenyl, and wherein phenyl is substituted or unsubstituted with up to five substituents independently selected from halogen, C₁-C₄alkyl, C₁-C₄ alkoxy, amino (wherein amino is optionally mono or disubstituted with C₁-C₄ alkyl), nitro or cyano; and X₁, X₂, X₃, X₄ and X₅ are independently hydrogen, fluorine, chlorine, bromine or iodine. Embodiments of the present invention include a compound of Formula 1 or Formula 2 wherein X₁, X₂, X₃, X₄ and X₅ are independently selected from hydrogen, fluorine, chlorine, bromine or iodine.

In certain embodiments, X₁, X₂, X₃, X₄ and X₅ are independently selected from hydrogen or chlorine. In other embodiments, X₁ is selected from fluorine, chlorine, bromine or iodine. In another embodiment, X₁ is chlorine, and X₂, X₃, X₄ and X₅ are hydrogen. In another embodiment, R₁, R₂, R₃ and R₄ are hydrogen.

The present invention provides enantiomers of Formula 1 or Formula 2 for treating epileptogenesis in a subject in need thereof. In certain embodiments, a compound of Formula 1 or Formula 2 will be in the form of a single enantiomer thereof. In other embodiments, a compound of Formula 1 or Formula 2 will be in the form of an enantiomeric mixture in which one enantiomer predominates with respect to another enantiomer.

In another aspect, one enantiomer predominates in a range of from about 90% or greater. In a further aspect, one enantiomer predominates in a range of from about 98% or greater.

The present invention also provides methods comprising administering to the subject a prophylactically or therapeutically effective amount of a composition that comprises at least one compound of Formula 1 or Formula 2 wherein R₁, R₂, R₃ and R₄ are independently selected from hydrogen or C₁-C₄ alkyl; and X₁, X₂, X₃, X₄ and X₅ are independently selected from hydrogen, fluorine, chlorine, bromine or iodine.

In embodiments of the present invention, before prophylactic or therapeutic administration of the composition to the subject, a determination will be made as to whether or not the subject suffers from epilepsy or an analogous seizure-related disorder or is considered to be at a high risk for the development of such seizures or seizure-related disorders.

The present invention also provides methods for identifying a subject in need of prophylactic or therapeutic administration of an anti-epileptogenic composition, wherein the subject suffers from epilepsy or an analogous seizure-related disorder or is considered to be at a high risk of developing epilepsy or wherein the subject is in need of treatment with an AEGD. The present invention provides methods comprising prophylactically or therapeutically administering to the subject a composition that comprises at least one compound having Formula 1 or Formula 2.

In certain embodiments of the present invention, a prophylactically or therapeutically effective amount of a compound of Formula 1 or Formula 2 for the treatment of epileptogenesis is in a range of from about 0.01 mg/Kg/dose to about 150 mg/Kg/dose.

In certain embodiments, a prophylactically or therapeutically effective amount of a pharmaceutical composition for preventing, treating, reversing, arresting or inhibiting epileptogenesis comprising one or more of the enantiomers of a compound of Formula 1 or Formula 2 includes a pharmaceutically acceptable salt or ester thereof in admixture with a pharmaceutically acceptable carrier or excipient, whereby such a composition is administered to the subject in need of treatment with an AEGD. Pharmaceutical compositions comprising at least one compound having Formula 1 or Formula 2 and one or more pharmaceutically acceptably excipients are administered to a subject in need thereof.

In certain embodiments, a subject or patient in need of treatment with an AEGD may be one who has not yet shown the symptoms of epilepsy or an analogous seizure-related disorder prior to the time of administration.

In another aspect, the subject or patient will be determined to be at risk for developing epilepsy or an analogous seizure-related disorder at the time of administration and will thereby be a subject, i.e., a patient in need of treatment with an AEGD. In other embodiments, the subject in need thereof is an individual who has shown the symptoms of epilepsy (e.g. overt seizures) or an analogous seizure-related disorder (e.g. mood cycling, impulsive behavior and the like) before or at the time of administration.

### BRIEF DESCRIPTION OF THE FIGURES

FIG. 1: is a graph that shows the effects of increasing doses of TC on the number of neurons in different areas of the hippocampus counted at 14 days after li-pilo SE. Values are expressed as the number of neuronal cell bodies in each area of interest ±S.E.M.

FIG. 2: is a graph that shows the effects of increasing doses of TC on the number of neurons in different nuclei of the amygdala counted at 14 days after li-pilo SE. Values are expressed as the number of neuronal cell bodies in each area of interest ±S.E.M.

FIG. 3: is a graph that shows the effects of increasing doses of TC on the number of neurons in different nuclei of the thalamus counted at 14 days after li-pilo SE. Values are expressed as the number of neuronal cell bodies in each area of interest ±S.E.M.

FIG. 4: is a graph that shows the effects of increasing doses of TC on the number of neurons in different areas of the cortex counted at 14 days after li-pilo SE. Values are expressed as the number of neuronal cell bodies in each area of interest ±S.E.M.

FIG. 5: is a graph that shows the effects of increasing doses of TC on the latency to the first spontaneous seizure. Values are expressed as the mean latency in days for each group ±S.E.M.

FIG. 6: is a graph that shows the effects of increasing doses of TC on the frequency of spontaneous seizures video-recorded over a 4 weeks period. Values are expressed as the mean number of seizures ±S.E.M. The total represents the total number of seizures observed during the 4 weeks of video-recording and the mean represents the mean number of seizures per week. The Anova test demonstrated an effect of the treatment on the total number of seizures (p=0.045) and the mean number of seizures per week (p=0.045)

FIG. 7: shows the total number of seizures video-recorded over four weeks plotted according to the latency to the first spontaneous seizure (SL=short latency, LL=long latency). Values are expressed as the mean number of seizures for each subgroup ±S.E.M. The ANOVA test did not show any significant effect of the treatment.

FIG. 8: shows the correlation between the latency to the first spontaneous seizure and the total number of seizures observed during the four following weeks.

### DETAILED DESCRIPTION OF THE INVENTION

The Carbamate Compounds of the Invention

The present invention provides methods of using 2-phenyl-1,2-ethanediol monocarbomates and dicarbamates in the treatment/prevention of epileptogenesis.

Representative carbamate compounds according to the present invention include those having Formula 1 or Formula 2: 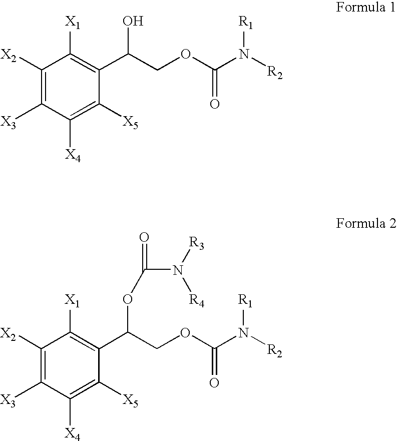
wherein:

R₁, R₂, R₃, and R₄ are, independently, hydrogen or C₁-C₄ alkyl and X₁, X₂, X₃, X₄, and X₅ are, independently, hydrogen, fluorine, chlorine, bromine or iodine.

“C₁-C₄ alkyl” as used herein refers to substituted or unsubstituted aliphatic hydrocarbons having from 1 to 4 carbon atoms. Specifically included within the definition of “alkyl” are those aliphatic hydrocarbons that are optionally substituted. In a preferred embodiment of the present invention, the C₁-C₄ alkyl is either unsubstituted or substituted with phenyl.

The term “phenyl”, as used herein, whether used alone or as part of another group, is defined as a substituted or unsubstituted aromatic hydrocarbon ring group having 6 carbon atoms. Specifically included within the definition of “phenyl” are those phenyl groups that are optionally substituted. For example, in a preferred embodiment of the present invention, the, “phenyl” group is either unsubstituted or substituted with halogen, C₁-C₄ alkyl, C₁-C₄ alkoxy, amino, nitro, or cyano.

In a preferred embodiment of the present invention, X₁ is fluorine, chlorine, bromine or iodine and X₂, X₃, X₄, and X₅ are hydrogen.

In another preferred embodiment of the present invention, X₁, X₂, X₃, X₄, and X₅ are, independently, chlorine or hydrogen.

In another preferred embodiment of the present invention, R₁, R₂, R₃, and R₄ are all hydrogen.

It is understood that substituents and substitution patterns on the compounds of the present invention can be selected by one of ordinary skill in the art to provide compounds that are chemically stable and that can be readily synthesized by techniques known in the art as well as the methods provided herein.

Representative 2-phenyl-1,2-ethanediol monocarbomates and dicarbamates include, for example, the following compounds:

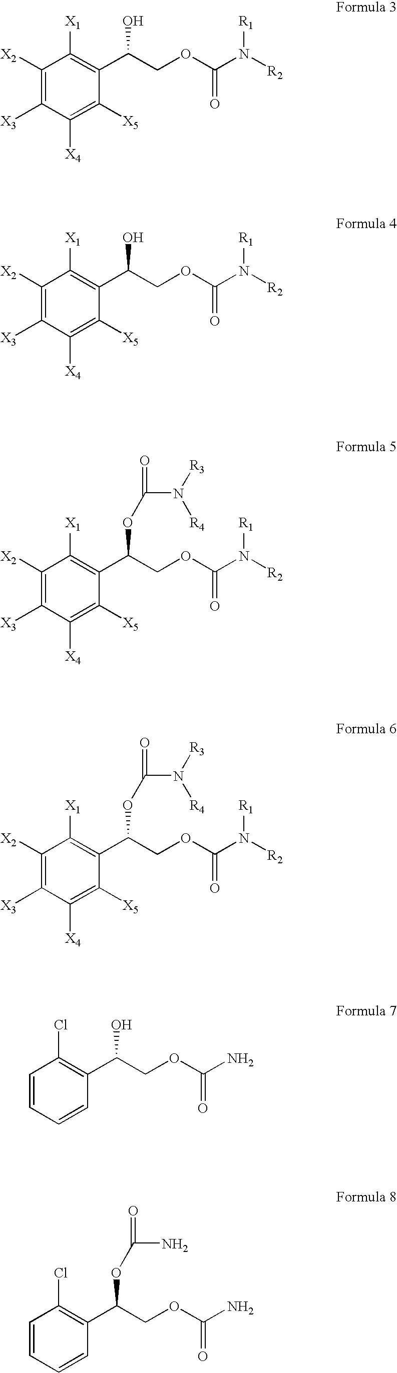

Suitable methods for synthesizing and purifying carbamate compounds, including carbamate enantiomers, used in the methods of the present invention are well known to those skilled in the art. For example, pure enantiomeric forms and enantiomeric mixtures of 2-phenyl-1,2-ethanediol monocarbomates and dicarbamates are described in U.S. Pat. Nos. 5,854,283, 5,698,588, and 6,103,759, the disclosures of which are herein incorporated by reference in their entirety.

The present invention includes the use of isolated enantiomers of Formula 1 or Formula 2.

In one preferred embodiment, a pharmaceutical composition comprising the isolated S-enantiomer of Formula 1 is used to treat epileptogenesis in a subject.

In another preferred embodiment, a pharmaceutical composition comprising the isolated R-enantiomer of Formula 2 is used to treat epileptogenesis in a subject.

In another embodiment, a pharmaceutical composition comprising the isolated S-enantiomer of Formula 1 and the isolated R-enantiomer of Formula 2 can be used to treat epileptogenesis in a subject.

The present invention also includes the use of mixtures of enantiomers of Formula 1 or Formula 2. In one aspect of the present invention, one enantiomer will predominate. An enantiomer that predominates in the mixture is one that is present in the mixture in an amount greater than any of the other enantiomers present in the mixture, e.g., in an amount greater than 50%. In one aspect, one enantiomer will predominate to the extent of 90% or to the extent of 91%, 92%, 93%, 94%, 95%, 96%, 97% or 98% or greater. In one preferred embodiment, the enantiomer that predominates in a composition comprising a compound of Formula 1 is the S-enantiomer of Formula 1. In another preferred embodiment, the enantiomer that predominates in a composition comprising a compound of Formula 2 is the R-enantiomer of Formula 2.

In a preferred embodiment of the present invention, the enantiomer that is present as the sole enantiomer or as the predominate enantiomer in a composition of the present invention is represented by Formula 3 or Formula 5, wherein X₁, X₂, X₃, X₄, X₅, R₁, R₂, R₃, and R₄ are defined as above, or by Formula 7 or Formula 8.

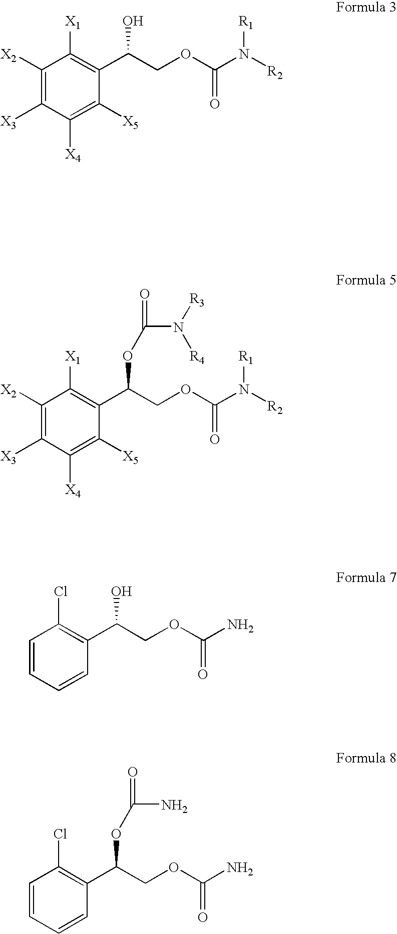

The present invention provides methods of using enantiomers and enantiomeric mixtures of compounds represented by Formula 1 and Formula 2 or a pharmaceutically acceptable salt or ester form thereof:

A carbamate enantiomer of Formula 1 or Formula 2 contains an asymmetric chiral carbon at the benzylic position, which is the aliphatic carbon adjacent to the phenyl ring.

An enantiomer that is isolated is one that is substantially free of the corresponding enantiomer. Thus, an isolated enantiomer refers to a compound that is separated via separation techniques or prepared free of the corresponding enantiomer. “Substantially free,” as used herein, means that the compound is made up of a significantly greater proportion of one enantiomer. In preferred embodiments, the compound includes at least about 90% by weight of a preferred enantiomer. In other embodiments of the invention, the compound includes at least about 99% by weight of a preferred enantiomer. Preferred enantiomers can be isolated from racemic mixtures by any method known to those skilled in the art, including high performance liquid chromatography (HPLC) and the formation and crystallization of chiral salts, or preferred enantiomers can be prepared by methods described herein.

Methods for the preparation of preferred enantiomers would be known to one of skill in the art and are described, for example, in Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N.Y., 1962); and Wilen, S. H. Tables of Resolving Agents and Optical Resolutions p. 268 (E. L. Eliel, Ed., Univ. of Notre Dame Press, Notre Dame, Ind. 1972). Additionally, compounds of the present invention can be prepared as described in U.S. Pat. No. 3,265,728 (the disclosure of which is herein incorporated by reference in its entirety and for all purposes), U.S. Pat. No. 3,313,692 (the disclosure of which is herein incorporated by reference in its entirety and for all purposes), and the previously referenced U.S. Pat. Nos. 5,854,283, 5,698,588, and 6,103,759 (the disclosures of which are herein incorporated by reference in their entirety and for all purposes).

The epileptogenic process generally consists of two phases. The first epileptogenic stage is known as the initial insult or injury stage. The initial insult or injury is commonly a brain-damaging injury caused by one or more of a multitude of possible factors including, for example, traumatic brain injury; CNS infection, such as, for example, bacterial meningitis, viral encephalitis, bacterial cerebral abscess or neurocysticercosis); cerebrovascular disease (such as stroke or brain tumor including, for example, malignant gliomas; neurosurgery (such as for example craniotomies); and status epilepticus.

In some instances, the initial insult will be a result of developmental problems before birth (such as, but not limited to, birth asphyxia, intracranial trauma during birth, metabolic disturbances or congenital malformations of the brain) or as the result of genetic determinants.

The second epileptogenic stage is known as the latency stage. The methods of the present invention include prophylactic or therapeutic administration of a carbamate compound of the present invention at either the first or second epileptogenic stage or preceding these stages to treat, inhibit, prevent, arrest or reverse the subsequent development of epilepsy or other analogous seizure-related disorder in a subject in need thereof.

The second epileptogenic stage further includes the process of neuronal restructuring, which is characterized by recurrent seizures (e.g. symptomatic epilepsy) or by symptoms shown in analogous seizure-related disorders. The epileptogenic process can also be observed among persons actually suffering from epilepsy or analogous seizure-related disorders. For example, seizures experienced by persons suffering from epilepsy are epileptogenic if they tend to make the occurrence of subsequent seizures more likely.

In a similar manner, the related seizure-like response in neurological or psychiatric disorders analogous to epilepsy may become increasingly severe over time or resistant to treatment as the disorder matures. The methods and compounds of the present invention are intended to be used to treat, prevent, arrest, inhibit or reverse the process of epileptogenesis in such analogous seizure-related neurological or psychiatric disorders as well.

In certain embodiments, phase 1 epileptogenesis can be initiated by factors other than those listed above, such as by the ingestion of compounds with epileptogenic potential, e.g., psychotropic medications such as, for example, tricyclic antidepressants, clozapine, and lithium and the like. The methods and compounds of the present invention are also intended to treat, prevent, arrest, inhibit or reverse the development of epileptogenesis which has been initiated by factors which tend to increase the potential for a subject to become epileptogenic.

Therefore, in treating epileptogenesis, the methods of the invention can forestall the development of seizures, particularly epileptic seizures. Such methods therefore can be used to treat and prevent epilepsy and epileptic seizures, reduce the risk of developing epilepsy, arrest the development of epilepsy (particularly, the development of collections of neurons which are the source of or are susceptible to ictogenic seizure), inhibit the development and maturation of epilepsy (particularly, the development of epileptogenic zones and epileptogenic focus), reduce the severity of epilepsy in a subject and reverse the process of epileptogenesis in epilepsy. In addition, by treating, preventing, inhibiting, arresting or reversing epileptogenesis according to the methods of the present invention, the development or progression of analogous of neurological and/or psychiatric disorders whose etiology is partly or wholly based on a seizure like mechanism of action will be treated, prevented, inhibited, arrested or reversed.

Definitions

As used herein, the term “epileptogenesis” means the biochemical, genetic, histological or other structural or functional processes or changes that make nervous tissue, including the central nervous system (CNS) susceptible to recurrent, spontaneous seizures. In addition, the term “epileptogenesis” is also used herein in a broader sense to refer to the changes and processes that contribute to the clinical progression observed in some epilepsies and the development of “pharmacoresistance,” in which epilepsy becomes more difficult to treat as a result of neurobiological changes which result in reduced drug sensitivity.

Furthermore the term “epileptogenesis” is used herein in the broadest possible sense to refer to the similar phenomena of progressive worsening over time of the signs and symptoms of apparently non-epileptic disorders, including psychiatric disorders the etiology of which appear to be seizure related. This is intended to include, but is not limited to, the worsening or progression of, for example: Bipolar Disorder over time or as a result of exposure to antidepressants or other drugs, as demonstrated by increased rate of cycling, increasing severity of episodes, increasingly severe psychotic symptoms and/or reduced responsiveness to treatment, etc.; Impulse Control Disorders; Obsessive-Compulsive Disorders, certain personality disorders, impulsive or aggressive behavior in neurodegenerative or related disorders.

The term “inhibition of epileptogenesis,” as used herein, refers to preventing, slowing, halting, or reversing the process of epileptogenesis.

The term “anti-epileptogenic agent or drug” (AEGD), as used herein, refers to an agent that is capable of inhibiting epileptogenesis when the agent is administered to a subject.

The term “convulsive disorder,” as used herein, refers to a disorder in a subject in which the subject suffers from convulsions, e.g., convulsions due to epileptic seizure. Convulsive disorders include, but are not limited to, epilepsy and non-epileptic convulsions, e.g., convulsions due to administration of a convulsive agent to the subject.

As used herein, the terms “analogous seizure-related disorder(s)” or “epilepsy related seizure like neurological phenomenon” refer to a neurobiological disorder or a psychiatric disorder that may show little or no overt seizure activity but which are still believed to be wholly or partly the result of a seizure-like or related neural mechanism and which are often found to be treatable with AEDs. Examples of analogous seizure-related disorder(s) include, but are not limited to; Bipolar Disorder, Schizoaffective Disorder, psychotic disorders, Impulse Control Disorders and the related impulse control disorder disease spectrum, eating disorders such as Bulimia or Anorexia Nervosa, Obsessive-Compulsive Disorder (OCD), substance abuse disorders, and the personality and behavioral changes that occur in patients with Temporal Lobe Epilepsy or in primary personality disorders.

As used herein, the term “subject” includes an individual or patient who has not yet shown the symptoms of epilepsy or analogous seizure-related disorder but who may be in a high risk group.

As used herein, the term “a subject in need of treatment with an AEGD” would include an individual who does not have epilepsy or analogous seizure-related disorder but who may be in a high-risk group for the development of seizures or a seizure related disorder because of injury or trauma to the CNS or PNS or one An individual or patient is considered to be at a high risk for the development of such seizures or seizure-related disorders because of injury or trauma to the CNS or PNS, because of some known biochemical or genetic predisposition to epilepsy or analogous seizure-related disorder, or because a verified biomarker or surrogate marker of one or more of these disorders has been discovered.

The term “a subject in need of treatment with an AEGD” would also include any individual whose clinical condition or prognosis could benefit from treatment with an AEGD. This would include, and is not limited to, any individual determined to be at an increased risk of developing epilepsy, a seizure disorder or analogous seizure-related disorder or epilepsy related seizure like neurological phenomenon or seizure related disorder as defined above, due to any predisposing factor. Predisposing factors include, but are not limited to: injury or trauma of any kind to the CNS; infections of the CNS, e.g., meningitis or encephalitis; anoxia; stroke, i.e., cerebro-vascular accidents (CVAs); autoimmune diseases affecting the CNS, e.g., lupus; birth injures, e.g., perinatal asphyxia; cardiac arrest; therapeutic or diagnostic vascular surgical procedures, e.g., carotid endarterectomy or cerebral angiography; heart bypass surgery; spinal cord trauma; hypotension; injury to the CNS from emboli, hyper or hypo perfusion of the CNS; hypoxia affecting the CNS; known genetic predisposition to disorders known to respond to AEGDs; space occupying lesions of the CNS; brain tumors, e.g., glioblastomas; bleeding or hemorrhage in or surrounding the CNS, e.g., intracerebral bleeds or subdural hematomas; brain edema; febrile convulsions; hyperthermia; exposure to toxic or poisonous agents; drug intoxication, e.g. cocaine; family history of seizure disorders or analogous seizure related disorder, history of status epilepticus; current treatment with medications that lower seizure threshold, e.g., lithium carbonate, thorazine or clozapine; evidence from surrogate markers or biomarkers that the patient is in need of treatment with an anti-epileptogenic drug, e.g. MRI scan showing hippocampal sclerosis or other CNS pathology, elevated serum levels of neuronal degradation products.

In addition, the term “a subject in need of treatment with an AEGD” would also etc. refer to any individual with a history of or who currently has; epilepsy, a seizure disorder or an analogous epilepsy related seizure like neurological phenomenon or seizure related disorder, as defined above, or any disorder in which the patient's present clinical condition or prognosis that could benefit from the suppression or inhibition of the process of epileptogenesis to prevent the extension, progression or increased resistance to treatment of any neurological or psychiatric disorder.

The term “antiepileptic drug” (AED) will be used interchangeably with the term “anticonvulsant agent,” and as used herein, both terms refer to an agent capable of inhibiting (e.g., preventing slowing, halting, or reversing) seizure activity or ictogenesis when the agent is administered to a subject or patient.

The term “pharmacophore” is known in the art, and, as used herein, refers to a molecular moiety capable of exerting a selected biochemical effect, e.g., inhibition of an enzyme, binding to a receptor, chelation of an ion, and the like. A selected pharmacophore can have more than one biochemical effect, e.g., can be an inhibitor of one enzyme and an agonist of a second enzyme. A therapeutic agent can include one or more pharmacophore, which can have the same or different biochemical activities.

The term “treating” or “treatment” as used herein, refers to actions that cause any indicia of success in the prevention or amelioration of an injury, pathology, symptoms or condition, including any objective or subjective parameters such as abatement; remission; diminishing of symptoms or making the injury, pathology, or condition more tolerable to the patient; slowing in the rate of degeneration or decline; making the final point of degeneration less debilitating; or improving a subject's physical or mental well-being.

Thus the term “treatment” or “to treat” is intended to include any action that improves, prevents, reverses, arrests, or inhibits the pathological process of epileptogenesis, as that term is defined and used herein. The treatment or amelioration of symptoms can be based on objective or subjective parameters; including the results of a physical examination, neurological examination, and/or psychiatric evaluations.

Accordingly, the term “treating” or “treatment” includes the administration of the compounds or agents of the present invention to treat, prevent, reverse, arrest, or inhibit the process of epileptogenesis. In some instances, treatment with the compounds of the present invention will prevent, inhibit, or arrest the progression of brain dysfunction or brain hyperexcitability associated with epilepsy.

The term “therapeutic effect” as used herein, refers to the treatment, inhibition, abatement, reversal, or prevention of epileptogenesis, symptoms of epileptogenesis, or side effects of epileptogenesis in a subject.

The term “a therapeutically effective amount” as used herein means a sufficient amount of one or more of the compounds of the invention to produce a therapeutic effect, as defined above, in a subject or patient in need of such treatment, inhibition, abatement, reversal, or prevention of epileptogenesis, symptoms of epileptogenesis, or side effects of epileptogenesis.

The terms “subject” or “patient” are used herein interchangeably and as used herein mean any mammalian subject or patient to whom the compositions of the invention can be administered. The term mammals include human patients and non-human primates, as well as experimental animals such as rabbits, rats, and mice, and other animals.

In some embodiments the methods of the present invention will be advantageously used to treat a patient who is not suffering or known to be suffering from a condition that is known in the art to be effectively treated with carbamate compounds or presently known AED's including analogous seizure-related disorder(s). In these cases the decision to use the methods and compounds of the present invention would be made on the basis of determining if the patient is a “patient in need of treatment with an anti-epileptogenic drug (AEGD)” as that term is defined above.

In some embodiments this invention provides methods of treating, preventing, reversing, arresting, or inhibiting epileptogenesis. In certain embodiments, these methods comprise administering a therapeutically effective amount of a carbamate compound to a patient who has not yet developed overt, clinical epilepsy or a seizure related disorder but who may be in a high risk group for the development of seizures or a seizure related disorder because of injury or trauma to the nervous system or because of some known predisposition either biochemical or genetic or the finding of a verified biomarker of one or more of these disorders.

Thus, in some embodiments, the methods and compositions of the present invention are directed toward treating epileptogenesis in a subject who is at risk of developing epilepsy or a seizure related disorder or analogous seizure related disorder(s) but who has not yet developed epilepsy or clinical evidence of seizures. A subject who is at risk of developing epilepsy or an analogous seizure related disorder(s) but who has not yet developed epilepsy or an analogous seizure related disorder(s) can be a subject who has not yet been diagnosed with epilepsy or an analogous seizure related disorder(s) but who is at greater risk than the general population for developing epilepsy or an analogous seizure related disorder(s). This “greater risk” may be determined by the recognition of any factor in a subject's, or their families, medical history, physical exam or testing that is indicative of a greater than average risk for developing epilepsy or an analogous seizure related disorder(s). Therefor this determination that a patient may be at a “greater risk” by any available means can be used to determine whether the patient should be treated with the methods of the present invention.

Accordingly, in an exemplary embodiments, subjects who may benefit from treatment by the methods and compounds of this invention can be identified using accepted screening methods to determine risk factors associated with epileptogenesis. These screening methods include, for example, conventional work-ups to determine risk factors that may be associated with epileptogenesis including but not limited to:, for example, head trauma, either closed or penetrating, CNS infections, bacterial or viral, cerebrovascular disease including but not limited to stroke, brain tumors, brain edema, cysticercosis, porphyria, metabolic encephalopathy, drug withdrawal including but not limited to sedative-hypnotic or alcohol withdrawal, abnormal perinatal history including anoxia at birth or birth injury of any kind, cerebral palsy, learning disabilities, hyperactivity, history of febrile convulsions as a child, history of status epilepticus, family history of epilepsy or any a seizure related disorder, inflammatory disease of the brain including lupis, drug intoxication either direct or by placental transfer, including but not limited to cocaine poisoning, parental consanguinity, and treatment with medications that lower seizure threshold including psychotropic medications.

In some embodiments the compounds of the present invention would be used for the manufacture of a medicament for the purpose of treating a patient in need of treatment with an anti-epileptogenic drug (AEGD). This would include the manufacture of a medicament for the purpose of treating a patient who currently had or was at risk of developing; epilepsy, a seizure disorder or an analogous seizure related disorder(s) or epilepsy related seizure like neurological phenomenon or seizure related disorder, as defined above, or any disorder in which the patient's present clinical condition or prognosis could benefit from the suppression or inhibition of the process of epileptogenesis to prevent the extension, worsening or increased resistance to treatment of any neurological or psychiatric disorder

The determination of which patients may benefit from treatment with an AEGD in patients who have no clinical signs or symptoms of epilepsy or related disorders may be based on a variety of “surrogate markers” or “biomarkers”.

As used herein, the terms “surrogate marker” and “biomarker” are used interchangeably and refer to any anatomical, biochemical, structural, electrical, genetic or chemical indicator or marker that can be reliably correlated with the present existence or future development of a seizure or seizure related disorder. In some instances, brain-imaging techniques, such as computer tomography (CT), magnetic resonance imaging (MRI) or positron emission tomography (PET), can be used to determine whether a subject is at risk for developing epilepsy or a seizure related disorder.

Suitable biomarkers for the methods of this invention include, but are not limited to: the determination by MRI, CT or other imaging techniques, of sclerosis, atrophy or volume loss in the hippocampus or overt mesial temporal sclerosis (MTS) or similar relevant anatomical pathology; the detection in the patient's blood, serum or tissues of a molecular species such as a protein or other biochemical biomarker, e.g., elevated levels of ciliary neurotrophic factor (CNTF) or elevated serum levels of a neuronal degradation product; or other evidence from surrogate markers or biomarkers that the patient is in need of treatment with an anti-epileptogenic drug, e.g. an EEG suggestive of a seizure disorder or an analogous seizure related disorder(s) epilepsy related seizure like neurological phenomenon or seizure related disorder.

It is expected that many more such biomarkers utilizing a wide variety of detection techniques will be developed in the future. It is intended that any such marker or indicator of the existence or possible future development of a seizure disorder or seizure related disorder, as the latter term is used herein, may be used in the methods of this invention for determining the need for treatment with the compounds and methods of this invention.

A determination that a subject has, or may be at risk for developing, epilepsy or an analogous seizure related disorder would also include, for example, a medical evaluation that includes a thorough history, a physical examination, and a series of relevant bloods tests. It can also include an electroencephalogram (EEG), CT, MRI or PET scan. A determination of an increased risk of developing an analogous seizure related disorder may also be made by means of genetic testing, including gene expression profiling or proteomic techniques. (See, Schmidt, D. Rogawski, M. A. Epilepsy Research 50; 71-78 (2002), and Loscher, W, Schmidt D. Epilepsy Research 50; 3-16 (2002))

For psychiatric disorders that may be “analogous seizure related disorders”, e.g., Bipolar Disorder, Impulse Control Disorders, etc. the above tests may also include a present state exam and a detailed history of the course of the patients symptoms such as mood disorder symptoms and psychotic symptoms over time and in relation to other treatments the patient may have received over time, e.g, a life chart. These and other specialized and routine methods allow the clinician to select patients in need of therapy using the methods and formulations of this invention.

In some embodiments of the present invention carbamate compounds suitable for use in the practice of this invention will be administered either singly or concomitantly with at least one or more other compounds or therapeutic agents, e.g., with other antiepileptic drugs, anticonvulsant drugs or neuroprotective drugs or electro convulsive therapy (ECT). In these embodiments, the present invention provides methods to treat, prevent or reverse epileptogenesis in a patient. The method includes the step of; administering to a patient in need of treatment, an effective amount of one of the carbamate compounds disclosed herein in combination with an effective amount of one or more other compounds or therapeutic agents that have the ability to treat or prevent epileptogenesis or the ability to augment the anti-epileptic or neuroprotective effects of the compounds of the invention.

As used herein, the term “concomitant administration” or “combination administration” of a compound, therapeutic agent or known drug with a compound of the present invention means administration of the drug and the one or more compounds at such time that both the known drug and the compound will have a therapeutic effect. In some cases this therapeutic effect will be synergistic. Such concomitant administration can involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compounds of the present invention.

The said one or more other compounds or therapeutic agents may be selected from compounds that have one or more of the following properties: antioxidant activity; NMDA receptor antagonist activity, augmentation of endogenous GABA inhibition; NO synthase inhibitor activity; iron binding ability, e.g., an iron chelator; calcium binding ability, e.g., a Ca (II) chelator; zinc binding ability, e.g., a Zn (II) chelator; the ability to effectively block sodium or calcium ion channels, or to open potassium or chloride ion channels in the CNS of a patient, such that epileptogenesis is inhibited in the patient.

In some preferred embodiments, the one or more other compounds or therapeutic agents would antagonize NMDA receptors by binding to the NMDA receptors (e.g., by binding to the glycine binding site of the NMDA receptors) and/or the agent would augment GABA inhibition by decreasing glial GABA uptake.

In addition the said one or more other compounds or therapeutic agents may be any agent known to suppress seizure activity even if that compound is not known to inhibit epileptogenesis. Such agents would include but not be limited to any effective AED known to one of skill in the art or discovered in the future, for example suitable agents include, but are not limited to; carbamazepine, clobazam, clonazepam, ethosuximide, felbamate, gabapentin, lamotigine, levetiracetam, oxcarbazepine, phenobarbital, phenytoin, pregabalin, primidone, retigabine, talampanel, tiagabine, topiramate, valproate, vigabatrin, zonisamide, benzodiazepines, barbiturates or sedative hypnotics.

In some embodiments of the present invention, treatment would be directed at patients who had epilepsy or an epilepsy related seizure like neurological phenomenon or an analogous seizure related disorder, as defined above, and by taking advantage of the ability of the compounds of the present invention to reverse epileptogenesis would allow the gradual reduction in the dosages of maintenance medication or intensity of treatment of being used to control the clinical manifestations of the patient's epilepsy or epilepsy related seizure like neurological phenomenon or analogous seizure related disorder, as defined above.

Therefore, as the treatment with the compounds of the invention produced improvement in the underlying disorder the patient could be withdrawn from their maintenance medication including but not limited to the compounds of the present invention themselves if they are being used as sole therapy. Thus, a patient with epilepsy on a maintenance therapy of a conventional AED could be withdrawn from the AED after the treatment with one or more of the compounds of the present invention had reversed the underlying epileptic disorder. In addition, a patient with an epilepsy related seizure like neurological phenomenon or an analogous seizure related disorder, as defined above, including but not limited to, for example, Bipolar Disorder, could be tapered off their maintenance medications, for example lithium carbonate, as treatment with one or more of the said compounds progressed. Likewise if one or more of the said compounds were being used as sole therapy the dose of this compound could be tapered to zero over time.

One of skill in the art could determine how rapidity to conduct the taper based on clinical signs and symptoms including EEG's, breakthrough seizures or other appropriate biomarkers of the underlying disorder.

Carbamate Compounds as Pharmaceuticals:

The present invention provides enantiomeric mixtures and isolated enantiomers of Formula 1 and/or Formula 2 as pharmaceuticals. The carbamate compounds are formulated as pharmaceuticals to treat epileptogenesis, e.g., to prevent, inhibit, reverse, or arrest the development of epilepsy in a subject.

In general, the carbamate compounds of the present invention can be administered as pharmaceutical compositions by any method known in the art for administering therapeutic drugs including oral, buccal, topical, systemic (e.g., transdermal, intranasal, or by suppository), or parenteral (e.g., intramuscular, subcutaneous, or intravenous injection.) Administration of the compounds directly to the nervous system can include, for example, administration to intracerebral, intraventricular, intacerebroventricular, intrathecal, intracisternal, intraspinal or peri-spinal routes of administration by delivery via intracranial or intravertebral needles or catheters with or without pump devices.

Compositions can take the form of tablets, pills, capsules, semisolids, powders, sustained release formulations, solutions, suspensions, emulsions, syrups, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, can be found in such standard references as Alfonso A R: Remington's Pharmaceutical Sciences, 17th ed., Mack Publishing Company, Easton Pa., 1985, the disclosure of which is incorporated herein by reference in its entirety and for all purposes. Suitable liquid carriers, especially for injectable solutions, include water, aqueous saline solution, aqueous dextrose solution, and glycols.

The carbamate compounds can be provided as aqueous suspensions. Aqueous suspensions of the invention can contain a carbamate compound in admixture with excipients suitable for the manufacture of aqueous suspensions. Such excipients can include, for example, a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate).

The aqueous suspension can also contain one or more preservatives such as ethyl or n-propyl p-hydroxybenzoate, one or more coloring agents, one or more flavoring agents, and one or more sweetening agents, such as sucrose, aspartame or saccharin. Formulations can be adjusted for osmolarity.

Oil suspensions for use in the present methods can be formulated by suspending a carbamate compound in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these.

Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations of the present invention suitable for parenteral administration, such as, for example, by intraarticular (in the joints), intravenous, intramuscular, intradermal, intraperitoneal, and subcutaneous routes, can include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter.

Where the compounds are sufficiently soluble they can be dissolved directly in normal saline with or without the use of suitable organic solvents, such as propylene glycol or polyethylene glycol. Dispersions of the finely divided compounds can be made-up in aqueous starch or sodium carboxymethyl cellulose solution, or in suitable oil, such as arachis oil. These formulations can be sterilized by conventional, well-known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The concentration of a carbamate compound in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally acceptable diluents or solvent, such as a solution of 1,3-butanediol. The formulations of commends can be presented in unit-dose or multi-dose sealed containers, such as ampoules and vials. Injection solutions and suspensions can be prepared from sterile powders, granules, and tablets of the kind previously described.

A carbamate compound suitable for use in the practice of this invention can be and is preferably administered orally. The amount of a compound of the present invention in the composition can vary widely depending on the type of composition, size of a unit dosage, kind of excipients, and other factors well known to those of ordinary skill in the art. In general, the final composition can comprise, for example, from 0.000001 percent by weight (% w) to 10% w of the carbamate compound, preferably 0.00001% w to 1% w, with the remainder being the excipient or excipients.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions.

Pharmaceutical preparations for oral use can be obtained through combination of the compounds of the present invention with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxymethyl cellulose, hydroxypropylmethyl-cellulose or sodium carboxymethylcellulose; and gums including arabic and tragacanth; as well as proteins such as gelatin and collagen. If desired, disintegrating or solubilizing agents can be added, such as the cross-linked polyvinyl pyrrolidone, agar, alginic acid, or a salt thereof, such as sodium alginate. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compounds of the present invention can also be administered in the form of suppositories for rectal administration of the drug. These formulations can be prepared by mixing the drug with a suitable non-irritating excipient that is solid at ordinary temperatures but liquid at the rectal temperatures and will therefore melt in the rectum to release the drug. Such materials are cocoa butter and polyethylene glycols.

The compounds of the present invention can also be administered by intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111,1995).

The compounds of the present invention can be delivered transdermally, by a topical route, formulated as applicator sticks, solutions, suspensions, emulsions, gels, creams, ointments, pastes, jellies, paints, powders, and aerosols.

Encapsulating materials can also be employed with the compounds of the present invention and the term “composition” can include the active ingredient in combination with an encapsulating material as a formulation, with or without other carriers. For example, the compounds of the present invention can also be delivered as microspheres for slow release in the body. In one embodiment, microspheres can be administered via intradermal injection of drug (e.g., mifepristone)-containing microspheres, which slowly release subcutaneously (see Rao, J. Biomater Sci. Polym. Ed. 7:623-645, 1995; as biodegradable and injectable gel formulations (see, e.g., Gao, Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months. Cachets can also be used in the delivery of the compounds of the present invention.

In another embodiment, the compounds of the present invention can be delivered by the use of liposomes which fuse with the cellular membrane or are endocytosed, i.e., by employing ligands attached to the liposome that bind to surface membrane protein receptors of the cell resulting in endocytosis. By using liposomes, particularly where the liposome surface carries ligands specific for target cells, or are otherwise preferentially directed to a specific organ, one can focus the delivery of the carbamate compound into target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587,1989).

The pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation can be a lyophilized powder which can contain, for example, any or all of the following: 1 mM-50 mM histidine, 0.1%-2% sucrose, 2%-7% mannitol, at a pH range of 4.5 to 5.5, that is combined with buffer prior to use.

Pharmaceutically acceptable salts and esters refers to salts and esters that are pharmaceutically acceptable and have the desired pharmacological properties. Such salts include salts that may be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N methylglucamine, and the like. Pharmaceutically acceptable salts can also include acid addition salts formed from the reaction of amine moieties in the parent compound with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds. When there are two acidic groups present, a pharmaceutically acceptable salt or ester may be a mono-acid-mono-salt or ester or a di-salt or ester; and similarly where there are more than two acidic groups present, some or all of such groups can be salified or esterified.

Compounds named in this invention can be present in unsalified or unesterified form, or in salified and/or esterified form, and the naming of such compounds is intended to include both the original (unsalified and unesterified) compound and its pharmaceutically acceptable salts and esters. The present invention includes pharmaceutically acceptable salt and ester forms of Formula 1 and Formula 2. More than one crystal form of an enantiomer of Formula 1 or Formula 2 can exist and as such are also included in the present invention.

A pharmaceutical composition of the invention can optionally contain, in addition to a carbamate compound, at least one other therapeutic agent useful in the treatment of a disease or condition associated with epileptogenesis.

Methods of formulating pharmaceutical compositions have been described in numerous publications such as Pharmaceutical Dosage Forms: Tablets. Second Edition. Revised and Expanded. Volumes 1-3, edited by Lieberman et al; Pharmaceutical Dosage Forms: Parenteral Medications. Volumes 1-2, edited by Avis et al; and Pharmaceutical Dosage Forms: Disperse Systems. Volumes 1-2, edited by Lieberman et al; published by Marcel Dekker, Inc, the disclosure of which are herein incorporated by reference in their entireties and for all purposes.

The pharmaceutical compositions are generally formulated as sterile, substantially isotonic and in full compliance with all Good Manufacturing Practice (GMP) regulations of the U.S. Food and Drug Administration.

Dosage Regimens

The present invention provides methods of treating epileptogenesis in a mammal using carbamate compounds. The amount of the carbamate compound necessary to treat a epileptogenesis is defined as a therapeutically or a pharmaceutically effective dose. The dosage schedule and amounts effective for this use, i.e., the dosing or dosage regimen will depend on a variety of factors including the stage of the disease, the patient's physical status, age and the like. In calculating the dosage regimen for a patient, the mode of administration is also taken into account.

A person of ordinary skill in the art will be able without undue experimentation, having regard to that skill and this disclosure, to determine a therapeutically effective amount of a particular substituted carbamate compound for practice of this invention (see, e.g., Lieberman, Pharmaceutical Dosage Forms(Vols. 1-3, 1992); Lloyd, 1999, The art, Science and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compounds.

For treatment purposes, the compositions or compounds disclosed herein can be administered to the subject in a single bolus delivery, via continuous delivery over an extended time period, or in a repeated administration protocol (e.g., by an hourly, daily or weekly, repeated administration protocol). The pharmaceutical formulations of the present invention can be administered, for example, one or more times daily, 3 times per week, or weekly. In one embodiment of the present invention, the pharmaceutical formulations of the present invention are orally administered once or twice daily.

A treatment regimen with the compounds of the present invention can commence, for example, after a subject suffers from a brain damaging injury or other initial insult but before the subject is diagnosed with epilepsy, e.g., before the subject has a first or second seizure. In one embodiment, a subject that is being treated with a compound having epileptogenic potential, e.g., psychotropic drug, or a subject having a disease associated with a risk of developing epilepsy, e.g., autism, can commence a treatment regimen with a carbamate compound of the present invention.

In certain embodiments, the carbamate compound can be administered daily for a set period of time (week, month, year) after occurrence of the brain damaging injury or initial insult. An attendant physician will know how to determine that the carbamate compound has reached a therapeutically effective level, e.g., clinical exam of a patient, or by measuring drug levels in the blood or cerebro-spinal fluid.

In this context, a therapeutically effective dosage of the biologically active agent(s) can include repeated doses within a prolonged treatment regimen that will yield clinically significant results to prevent, reverse, arrest, or inhibit the development of epilepsy. Determination of effective dosages in this context is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

In an exemplary embodiment of the present invention, unit dosage forms of the compounds are prepared for standard administration regimens. In this way, the composition can be subdivided readily into smaller doses at the physician's direction. For example, unit dosages can be made up in packeted powders, vials or ampoules and preferably in capsule or tablet form.

The active compound present in these unit dosage forms of the composition can be present in an amount of, for example, from about 10 mg. to about one gram or more, for single or multiple daily administration, according to the particular need of the patient. By initiating the treatment regimen with a minimal daily dose of about one gram, the blood levels of the carbamate compounds can be used to determine whether a larger or smaller dose is indicated.

Effective administration of the carbamate compounds of this invention can be administered, for example, at an oral or parenteral dose of from about 0.01 mg/kg/dose to about 150 mg/kg/dose. Preferably, administration will be from about 0.1/mg/kg/dose to about 25 mg/kg/dose, more preferably from about 0.2 to about 18 mg/kg/dose. Therefore, the therapeutically effective amount of the active ingredient contained per dosage unit as described herein can be, for example, from about 1 mg/day to about 7000 mg/day for a subject having, for example, an average weight of 70 kg.

Kits for use in Treating Epileptogenesis

After a pharmaceutical comprising a carbamate compound has been formulated in a suitable carrier, it can be placed in an appropriate container and labeled for treatment of epileptogenesis. Additionally, another pharmaceutical comprising at least one other therapeutic agent useful in the treatment of epileptogenesis, epilepsy or another disorder or condition associated with epileptogenesis can be placed in the container as well and labeled for treatment of the indicated disease. Such labeling can include, for example, instructions concerning the amount, frequency and method of administration of each pharmaceutical.

Although the foregoing invention has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. The following examples are provided to illustrate specific aspects of the invention and are not meant to be limitations.

### EXAMPLES

The activity of an isolated S-enantiomer of Formula 1 (e.g., Formula 7), herein referred to as the “test compound” or TC, was evaluated in the following experiments to determine the efficacy of the compound for neuroprotection and in the treatment of epileptogenesis in the model of temporal lobe epilepsy induced by lithium and pilocarpine in the rat.

### Example 1

The lithium-pilocarpine Model of Temporal Lobe Epilepsy

The model induced in rats by pilocarpine associated with lithium (Li-Pilo) reproduces most of the clinical and neurophysiological features of human temporal lobe epilepsy (Turski et al., 1989, Synapse 3:154-171; Cavalheiro, 1995, Ital J Neurol Sci 16:33-37). In adult rats, the systemic administration of pilocarpine leads to status epilepticus (SE). The lethality rate reaches 30-50% during the first days. In the surviving animals, neuronal damage predominates within the hippocampal formation, the piriform and entorhinal cortices, thalamus, amygdaloid complex, neocortex and substantia nigra. This acute seizure period is followed by a “silent” seizure-free phase lasting for a mean duration of 14-25 days after which all animals exhibit spontaneous recurrent convulsive seizures at the usual frequency of 2 to 5 per week (Turski et al., 1989, Synapse 3:154-171; Cavalheiro, 1995, Ital J Neurol Sci 16:33-37; Dube et al., 2001, Exp Neurol 167:227-241).

Lithium-pilocarpine and Treatments with the Test Compound

Male Wistar rats weighing 225-250 g, provided by Janvier Breeding Center (Le Genest-St-lste, France) were housed under controlled standard conditions (light/dark cycle, 7.00 a.m.-7.00 p.m. lights on), with food and water available ad libitum. All animal experimentation was performed in accordance with the rules of the European Communities Council Directive of Nov. 24, 1986 (86/609/EEC), and the French Department of Agriculture (License N^{o} 67-97). For electrode implantation, rats were anesthetized by an i.p. injection of 2.5 mg/kg diazepam (DZP, Valium, Roche, France) and 1 mg/kg ketamine chlorhydrate (Imalgene 1000, Rhone Merrieux, France). Four single-contact recording electrodes were placed on the skull, over the parietal cortex, two on each side.

Status Epilepticus Induction:

Treatment with the Test Compound and Occurrence of Spontaneous Recurrent Seizures (SRS)

All rats received lithium chloride (3 meq/kg, i.p., Sigma, St Louis, Mo., U.S.A.); about 20 h later, animals were placed into plexiglas boxes, in order to record baseline cortical EEG. Methylscopolamine bromide (1 mg/kg, s.c., Sigma) was administered to limit the peripheral effects of the convulsant. SE was induced by injecting pilocarpine hydrochloride (25 mg/kg, s.c., Sigma) 30 min after methyl-scopolamine. The bilateral EEG cortical activity was recorded during the whole duration of SE and behavioral changes were noted.

The effects of increasing doses of the test compound were studied on 3 groups of rats. The animals of the first group received 10 mg/kg of the test compound, i.p., 1 h after the onset of SE (pilo-TC10) while the animals of groups 2 and 3 received 30 and 60 mg/kg of the test compound (pilo-TC30 and pilo-TC60), respectively.

Another group was injected with 2 mg/kg diazepam (DZP, i.m.) at 1 h after the onset of SE which are our standard treatment to improve animals survival after SE (pilo-DZP). The control group received saline instead of pilocarpine and the test compound (saline-saline). The pilo-test compound rats surviving SE were then injected about 10 h after the first test compound injection with a second i.p. injection of the same dose of the test compound and were maintained under a twice daily treatment with the test compound for 6 additional days. Pilo-DZP received a second injection of 1 mg/kg DZP on the day of SE at about 10 h after the first one. Thereafter, Pilo-DZP and saline-saline rats received twice daily an equivalent volume of saline.

The effects of the test compound on the EEG and on the latency to occurrence of SRS were investigated by daily video recording of the animals for 10 h per day and the recording of the electrographic activity twice a week for 8 h.

Quantification of Cell Densities

Quantification of cell densities was performed at 6 days after SE on 8 pilo-DZP, 8 pilo-TC10, 7 pilo-TC30, 7 pilo-TC60, and 6 saline-saline rats. At 14 days after SE, animals were deeply anesthetized with 1.8 g/kg pentobarbital (Dolethal®, Vetoquinol, Lure, France. Brains were then removed and frozen. Serial 20 μm slices were cut in a cryostat, air-dried during several days before thionine staining.

Quantification of cell densities was performed with a 10×10 boxes 1 cm² microscopic grid on coronal sections according to the stereotaxic coordinates of the rat brain atlas (Paxinos and Watson, 1986). Cell counts were performed twice in a blind manner and were the average of at least 3 values from 2 adjacent sections in each individual animal. Counts involved only cells larger than 10 μm, smaller ones being considered as glial cells.

Timm staining

At 2 months after the onset of spontaneous recurrent seizures, mossy fiber sprouting was examined on rats in the chronic period exposed to the test compound or DZP and in 3 saline-saline rats. Animals were deeply anaesthetized and perfused transcardially with saline followed by 100 ml of 1.15% (w/v) Na₂S in 0.1 M phosphate buffer, and 100 ml of 4% (v/v) formaldehyde in 0.1 M phosphate buffer. Brains were removed from skull, post-fixed in 4% formaldehyde during 3-5 h and 40 μm sections were cut on a sliding vibratome and mounted on gelatin-coated slides.

The following day, sections were developed in the dark in a 26° C. solution of 50% (w/v) arabic gum (160 ml), sodium citrate buffer (30 ml), 5.7% (w/v) hydroquinone (80 ml) and 10% (w/v) silver nitrate (2.5 ml) during 40-45 min. The sections were then rinsed with tap water at 40° C. during at least 45 min, rinsed rapidly with distilled water and allowed to dry. They were dehydrated in ethanol and coverslipped.

Mossy fiber sprouting was evaluated according to criteria previously described in dorsal hippocampus (Cavazos et al., 1991, J Neurosci 11:2795-2803.), which are follows: 0—no granules between the tips and crest of the DG; 1—sparse granules in the supragranular region in a patchy distribution between the tips and crest of DG; 2—more numerous granules in a continuous distribution between the tips and crest of DG; 3—prominent granules in a continuous pattern between tips and crest, with occasional patches of confluent granules between tips and crest; 4—prominent granules that form a confluent dense laminar band between tips and crest and 5—confluent dense laminar band of granules that extends into the inner molecular layer.

Data Analysis

For the comparison of the characteristics of SE in pilo-saline and pilo-test compound animals, a non-paired Student's t-test was used. The comparison between the number of rats seizing in both groups was performed by means of a Chi square test. For neuronal damage, statistical analysis between groups was performed using ANOVA followed by a Fisher's test for multiple comparisons using the Statview software (Fisher R A, 1946a, Statistical Methods for Research Workers (10th edition) Oliver & Boyd, Edinburgh; Fisher R A, 1946b, The Design of Experiments (4th edition) Oliver & Boyd, Edinburgh) Behavioral and EEG Characteristics of Lithium-pilocarpine Status Epilepticus

A total number of Sprague-Dawley rats weighing 250-330 g were subjected to Li-pilo induced SE. The behavioral characteristics of SE were identical in both pilo-saline and pilo-test compound groups. Within 5 min after pilocarpine injection, rats developed diarrhea, piloerection and other signs of cholinergic stimulation. During the following 15-20 min, rats exhibited head bobbing, scratching, chewing and exploratory behavior. Recurrent seizures started around 15-20 min after pilocarpine administration. These seizures which associated episodes of head and bilateral forelimb myoclonus with rearing and falling progressed to SE at about 35-40 min after pilocarpine, as previously described (Turski et al., 1983, Behav Brain Res 9:315-335.).

EEG Patterns During SE

During the first hour of SE, in the absence of pharmacological treatment, the amplitude of the EEG progressively increased while the frequency decreased. Within 5 min after the injection of pilocarpine, the normal background EEG activity was replaced with low voltage fast activity in the cortex while theta rhythm (5-7 Hz) appeared in the hippocampus. By 15-20 min, high voltage fast activity superposed over the hippocampal theta rhythm and isolated high voltage spikes were recorded only in the hippocampus while the activity of the cortex did not substantially change.

By 35-40 min after pilocarpine injection, animals developed typical electrographic seizures with high voltage fast activity present in both the hippocampus and cortex which first occurred as bursts of activity preceding seizures and were followed by continuous trains of high voltage spikes and polyspikes lasting until the administration of DZP or the test compound. At about 3-4 h of SE, the hippocampal EEG was characterized by periodic electrographic discharges (PEDs, about one/sec) in the pilo-DZP and in the pilo-10 group in both hippocampus and cortex. The amplitude of EEG background activity was low in the pilo-TC60 animals. By 6-7 h of SE, spiking activity was still present in the cortex and the hippocampus in the DZP-and TC10-treated rats while the amplitude of the EEG decreased and came back to baseline levels in the hippocampus of TC30 rats and in both structures of TC60 treated rats. There was no difference between TC10, TC30, and TC60 groups. By 9 h of SE, isolated spikes were still recorded in the hippocampus of test compound-treated rats and occasionally in the cortex. In both structures, the background activity was of very low amplitude at that time.

Mortality Induced by SE

During the first 48 h after SE, the degree of mortality was similar in pilo-DZP rats (23%, 5/22), pilo-TC10 rats (26%, 6/23), and pilo-TC30 rats (20%, 5/25), The mortality rate was largely reduced in pilo-TC60 rats in which it only reached 4% (1/23). The difference was statistically significant (p<0.01).

EEG Characteristics of the Silent Phase and Occurrence of Spontaneous Recurrent Seizures

The EEG patterns during the silent period were similar in pilo-DZP and pilo-TC10, 30 or 60 rats. At 24 and 48 h days after SE, the baseline EEG was still characterized by the occurrence of PEDs on which large waves or spikes could be superimposed. Between 1 and 8 h after injection of the test compound or vehicle injection, there was no change in the pilo-DZP or pilo-TC10 groups. In TC30 and TC60 rats, the frequency and amplitude of PEDs decreased as soon as 10 min after injection and were replaced by spikes of large amplitude in the TC30 group and of low amplitude in the TC60 group. By 4 h after injection the EEG had returned to baseline levels in the two latter groups. By 6 days after SE, the EEG was still of lower amplitude than before pilocarpine injection and in most groups spikes could still be recorded, occasionally in the pilo-DZP, -TC10 and -TC30 rats. In pilo-TC60 rats, the frequency of large amplitude spikes was higher than in all other groups. After the test compound or vehicle-injection, EEG recording was not affected by the injection in the pilo-DZP and pilo-TC10 groups. In pilo-TC30 rats, the injection induced the occurred of slow waves on the EEG of both hippocampus and cortex and a decreased frequency of spikes in the pilo-TC60 rats.

All the rats exposed to DZP, TC10 and TC30 and studied until the chronic phase developed SRS with a similar latency. The latency was 18.2±6.9 days (n=9) in pilo-DZP rats, 15.4±5.1 days (n=7) in pilo-TC10 rats, 18.9±9.0 days (n=10) in pilo-TC30 rats. In the group of rats subjected to TC60, a subgroup of rats became epileptic with a latency similar to that of the other groups, i.e. 17.6±8.7 days (n=7) while another group of rats became epileptic with a much longer delay ranging from 109 to 191 days post-SE (149.8±36.0 days, n=4) and one rat did not become epileptic in a delay of 9 months post-SE. The difference in the latency to SRS between pilo-DZP, pilo-TC10, pilo-TC30 and the first subgroup of pilo-TPM60 rats was not statistically significant. None of the saline-saline rats (n=5) developed SRS.

To calculate the frequency of SRS in pilocarpine-exposed rats, the seizure severity and distinguished stage III (clonic seizures of facial muscles and anterior limbs) and stage IV-V seizures (rearing and falling) was considered. The frequency of stage III SRS per week in pilo-DZP and pilo-test compound rats was variable amongst the groups. It was low, constant in the pilo-DZP and pilo-TC60 (with early SRS onset) groups during the first 3 weeks and had disappeared during the 4th week in the pilo-DZP group. The frequency of stage III SRS was higher in the pilo-TC10 group where it was significantly increased over pilo-DZP values during weeks 3 and 4. The frequency of more severe stage IV-V SRS was highest during the first week in most groups, except pilo-TC30 and TC60 with late seizure onset where the SRS frequency was constant over the whole 4 weeks in TC30 group and over the first two weeks in the pilo-TC60 group with late SRS onset in which no stage IV-V seizures where no seizures recorded after the second week. The frequency of stage IV-V SRS was significantly reduced in the TC10, TC30 and TC60 (with early SRS onset) groups (2.3-6,1 SRS per week) compared to the pilo-DZP group (11.3 SRS per week) during the first week. During weeks 2-4v the frequency of stage IV-V SRS was reduced in all groups compared to the first week reaching values of 2-6 seizures per week, except in the pilo-TC60 group with early SRS onset where the frequency of seizures was significantly reduced to 0.6-0.9 seizure per week compared to the pilo-DZP group in which the frequency of SRS ranged from 3.3 to 5.8.

Cell Densities in Hippocampus, Thalamus and Cortex

In pilo-DZP rats compared to saline-saline rats, the number of cells was massively decreased in the CA1 region of the hippocampus (70% cell loss in the pyramidal cell layer) while the CAS region was less extensively damaged (54% cell loss in CA3a and 31% in CA3b). In the dentate gyrus, the pilo-DZP rats experienced extensive cell loss in the hilus (73%) while the granule cell layer did not show visible damage. Similar damage was observed in the ventral hippocampus but cell counts were not performed in this region. Extensive damage was also recorded in the lateral thalamic nucleus (91% cell loss) while the mediodorsal thalamic nucleus was more moderately damaged (56%). In the piriform cortex, cell loss was total in layers III-IV which was no longer visible and reached 53% in layer II in pilo-DZP rats. In the dorsal entorhinal cortex, layers II and III-IV underwent slight damage (9 and 15%, respectively). Layer II of the ventral entorhinal cortex was totally preserved while layers III-IV underwent a 44% cell loss.

In the hippocampus of pilo-test compound animals, cell loss was reduced compared to pilo-DZP rats in the CA1 pyramidal layer in which the cell loss reached 75% in pilo-DZP and 35 and 16% in the pilo-TC30 or pilo-TC60 animals, respectively. This difference was statistically significant at the two test compound doses. In the CAS pyramidal layer, the test compound did not afford any protection in the CA3a area while the 60 mg/kg of the test compound dose was significantly neuroprotective in CA3b. In the dentate gyrus, the cell loss in the hilus was similar in pilo-test compound (69-72%) and pilo-DZP animals (73%). In the two thalamic nuclei, the 60 mg/kg dose was also protective in reducing neuronal damage by 65 and 42% in the lateral and mediodorsal nucleus, respectively. In the cerebral cortex, the treatment with the test compound afforded neuronal protection compared to DZP only at the highest dose, 60 mg/kg. At the two lowest doses, 10 and 30 mg/kg, the total loss of cells and tissue disorganization observed in layers III-IV of the piriform cortex was identical in pilo-DZP rats and pilo-test compound rats and did not allow any counting in any of the groups. In layers II and III-IV of the piriform cortex, the TC60 treatment reduced neuronal damage recorded in the pilo-DZP rats by 41 and 44%, respectively. In the ventral entorhinal cortex, neuroprotection was induced by TC60 administration in layers III-IV and reached 31% compared to pilo-DZP rats. In the entorhinal cortex, there was a slight worsening of cell loss in pilo-TC10 rats compared with pilo-DZP rats in layers III-IV of the dorsal entorhinal cortex (28% more damage) and layers III-IV of the ventral entohinal cortex (35% more damage). At the other doses of the test compound, cell loss in the entorhinal cortex was similar to the one recorded in pilo-DZP rats.

Mossy Fiber Sprouting in Hippocampus

All rats exhibiting SRS in pilo-DZP and pilo-TPM groups showed similar intensity of Timm staining in the inner molecular layer of the dentate gyrus (scores 2-4). Timm staining was present both on the upper and lower blades of the dentate gyrus. The mean value of the Timm score in the upper blade reached 2.8±0.8 in pilo-DZP rats (n=9), 1.5±0.6 in pilo-TC10 rats (n=7), 2.6±1.0 in pilo-TC30 rats (n=10), and 1.5±0.7 in the whole group of pilo-TC60 rats (n =11). When the pilo-test compound at 60mg/kg.group was subdivided according to the latency to SRS, the subgroup with early SRS occurrence showed a Timm score of 1.8±0.6 (n=6) and the subgroup of rats with late occurrence or absence of SRS had a Timm score of 1.2±0.6 (n=5). The values recorded in the pilo-DZP rats were statistically significantly different from the values in the pilo-TC10 (p=0.032) and the pilo-TC60 subgroup with late or no seizures (p=0.016).

Discussion and Conclusions

The results of the present study show that a 7-day treatment with the test compound starting at 1 h after the onset of SE is able to protect some brain areas from neuronal damage, e.g., in the pyramidal cell layer of the CA1 and CA3b area, the mediodorsal thalamus, layers II and I1MV of the piriform cortex and layers III-IV of the ventral entorhinal cortex, but only at the highest dose the test compound, i.e. 60 mg/kg. The latter dose of the test compound is also able to delay the occurrence of SRS, at least in a subgroup of animals that became epileptic with a mean delay that was about 9-fold longer than in the other groups of animals and one animal did not become epileptic in a delay of 9 months after SE.

These results show that one compound with anti-ictal properties, which are the classical properties of most antiepileptic-marketed drugs, is also able to delay epileptogenesis, i.e. to be antiepileptogenic. The data of the present study show also that the test compound treatment, whatever the dose used, decreases the severity of the epilepsy since it decreases the number of stage IV-V seizures, mainly during the first week of occurrence and during the whole period of 4-weeks observation with the test compound at 60mg/kg. treatment. Moreover, in the TC10 group, there is a shift to an increase in the occurrence of less severe stage III seizures that are more numerous than in the pilo-DZP group.

### Example Two

The aim of the present project was to pursue the study reported in Example one above on the potential neuroprotective and antiepileptogenic properties of the test compound (TC) in the lithium-pilocarpine (Li-Pilo) model of temporal lobe epilepsy. In the first study it was shown that TC was able to protect areas CA1 and CA3 of the hippocampus, piriform and ventral entorhinal cortex from neuronal damage induced by Li-Pilo status epilepticus (SE). Most of these neuroprotective properties occurred at the highest dose studied, 60 mg/kg and the treatment was able to delay the occurrence of spontaneous seizures in 36% (4 out of 11) of the rats. In the present example, the consequences of treatment by higher doses of TC on neuronal damage and epileptogenesis is studied

The Lithium-pilocarpine Model of Temporal Lobe Epilepsy

The model of epilepsy induced in rats by pilocarpine associated with lithium (Li-Pilo) reproduces most of the clinical and neurophysiological features of human temporal lobe epilepsy (Turski et al., 1989; Cavalheiro, 1995). In adult rats, the systemic administration of pilocarpine leads to SE which may last for up to 24 h. The lethality rate reaches 30-50% during the first days. In the surviving animals, neuronal damage predominates within the hippocampal formation, the piriform and entorhinal cortices, thalamus, amygdaloid complex, neocortex and substantia nigra. This acute seizure period is followed by a “silent” seizure-free phase lasting for a mean duration of 14-25 days after which all animals exhibit spontaneous recurrent convulsive seizures at the usual frequency of 2 to 5 per week (Turski et al., 1989; Cavalheiro, 1995; Dubé et al., 2001). The current antiepileptic drugs do not prevent the epileptogenesis and are only transiently efficient on recurrent seizures.

In our previous study, we studied the potential neuroprotective and antiepileptogenic effects of increasing doses of TC given in monotherapy and compared to our standard diazepam (DZP) treatment mostly given to prevent high mortality. These data show that a 7-day treatment with 10, 30 or 60 mg/kg TC starting at 1 h after the onset of SE is able to protect some brain areas from neuronal damage. This effect is statistically significant in the pyramidal cell layer of the CA1 and CA3b area, the mediodorsal thalamus, layers II and III-IV of the piriform cortex and layers III-IV of the ventral entorhinal cortex, but only at the highest dose of TC, i.e. 60 mg/kg. Moreover, it appears that the latter dose of TC is also the only one that is able to delay the occurrence of SRS, at least in a subgroup of animals that became epileptic with a mean delay that was about 9-fold longer than in the other groups of animals and one animal did not become epileptic in a delay of 9 months after SE.

In the present study, the effects of different doses of TC, i.e. 30, 60, 90 and 120 mg/kg (TC30, TC60, TC90 and TC120) were tested using the same design as in the previous study. The treatment was started one hour after the onset of SE and the animals were treated with a second injection of the same dose of the drug. This early treatment of SE was followed by a 6 days TC treatment. This report concerns the effects of the four different doses of TC on neuronal damage assessed in hippocampus, parahippocampal cortices, thalamus and amygdala at 14 days after SE and on the latency to and frequency of spontaneous epileptic seizures.

Methods

Animals

Adult male Sprague-Dawley rats provided by Janvier Breeding Center (Le Genest-St-lsle, France) were housed under controlled, uncrowded standard conditions at 20-22° C. (light/dark cycle, 7.00 a.m.-7.00 p.m. lights on), with food and water available ad libitum. All animal experimentation was performed in accordance with the rules of the European Communities Council Directive of Nov. 24, 1986 (86/609/EEC), and the French Department of Agriculture (License N^{o} 67-97).

Status Epilepticus Induction, TC Treatment and Occurrence of SRS

All rats received lithium chloride (3 meq/kg, i.p., Sigma, St Louis, Mo., U.S.A.) and about 20 h later, all animals received also methylscopolamine bromide (1 mg/kg, s.c., Sigma) that was administered to limit the peripheral effects of the convulsant. SE was induced by injecting pilocarpine hydrochloride (25 mg/kg, s.c., Sigma) 30 min after methylscopolamine. The effects of increasing doses of TC were studied in 5 groups of rats. The animals received either 2.5 mg/kg DZP, i.m., or 30, 60, 90 or 120 mg/kg TC (TC30, TC60, TC90, TC120), i.p., at 1 h after the onset of SE. The control group received vehicle instead of pilocarpine and TC. The rats surviving SE were then injected about 10 h after the first TC injection with a second i.p. injection of 1.25 mg/kg DZP for the DZP group or of the same dose of TC as in the morning and were maintained under a twice daily TC treatment (s.c.) for 6 additional days while DZP rats received a vehicle injection.

The effects of DZP and the 4 doses of TC on epileptogenesis were investigated by daily video recording of the animals for 10 h per day. Video recording was performed for 4 weeks during which the occurrence of the first seizure was noted as well as the total number of seizures over the whole period. Animals were then taken off the video recording system and kept for 4 additional weeks in our animal facilities before they were sacrificed after a total period of 8 weeks of epilepsy. The rats that did not exhibited seizures were sacrificed after 5 months of video recording.

Quantification of Cell Densities

Quantification of cell densities was performed at two times after SE: a first group was studied 14 days after SE and was composed by 7 DZP, 8 TC30, 11 TC60, 10 TC90 , 8 TC120 and 8 control rats not subjected to SE. A second group used for the study of the latency to SRS was sacrificed either 8 weeks after the first SRS or at 5 months when no SRS could be seen in that delay and was composed by 14 DZP, 8 TC30, 10 TC60, 11 TC90, 9 TC120 rats. At the moment, neuronal counting is still in progress in the second group of animals studied for epileptogenesis and long-term counting and the data concerning that part of the study will not be included in the present report.

For neuronal counting, animals were deeply anesthetized with 1.8 g/kg pentobarbital (Dolethal®, Vétoquinol, Lure, France). Brains were then removed and frozen. Serial 20 μm slices were cut in a cryostat, air-dried during several days before thionine staining. Quantification of cell densities was performed with a 10×10 boxes 1 cm² microscopic grid on coronal sections according to the stereotaxic coordinates of the rat brain atlas (Paxinos and Watson, 1986). The grid of counting was placed on a well defined area of the cerebral structure of interest and counting was carried out with a microscopic enlargement of 200- or 400-fold defined for each single cerebral structure. Cell counts were performed twice on each side of three adjacent sections for each region by a single observer unaware of the animal's treatment. The number of cells obtained in the 12 counted fields in each cerebral structure was averaged. This procedure was used to minimize the potential errors that could result from double counting leading to overestimation of cell numbers. Neurons touching the inferior and right edges of the grid were not counted. Counts involved only neurons with cell bodies larger than 10 μm. Cells with small cell bodies were considered as glial cells and were not counted.

Data Analysis

For neuronal damage and epileptogenesis, statistical analysis between groups was performed by means of a one-way analysis of variance followed by a post-hoc Dunnett or Fisher test using the Statistica software.

Results

Behavioral Characteristics of Lithium-pilocarpine Status Epilepticus

A total number of 143 Sprague-Dawley rats weighing 250-330 g were subjected to lithium-pilocarpine (Li-pilo)-induced SE. In this number 10 did not develop SE while 133 rats developed a full characteristic Li-pilo SE. The behavioral characteristics of SE were identical in both li-pilo-DZP and li-pilo-TC groups. Within 5 min after pilocarpine injection, rats developed diarrhea, piloerection and other signs of cholinergic stimulation. During the following 15-20 min, rats exhibited head bobbing, scratching, chewing and exploratory behavior. Recurrent seizures started around 15-20 min after pilocarpine administration. These seizures which associated episodes of head and bilateral forelimb myoclonus with rearing and falling progressed to SE at about 35-40 min after pilocarpine, as previously described (Turski et al., 1989; Dubé et al., 2001; André et al., 2003). The control group not subjected to SE and receiving lithium and saline was composed of 20 rats.

In the group of 57 animals devoted to cell counting at 14 days after SE, a total number of 13 rats died over the first 48 h after SE. The degree of mortality varied with the treatment: 36% (4/11) of DZP rats, 33% (4/12) of TC30 rats, 8% (1/12) of TC60 rats, 0% (0/10) of TC90 rats and 33% (4/12) of TC120 rats died. In the DZP group, the 4 rats died in the first 24 h after SE. In the group of TC30 rats, one rat died on the day of SE, one rat was dead by 24 h after SE and two rats by 48 h. In the group of TC60 rats, one rat died at 48 h after SE. In the group of TC120 rats, two rats were dead by 24 h and two by 48 h after SE.

In the group of 55 animals devoted to the study of the latency to SRS and late cell counting, the degree of mortality over the first 48 h after SE was the following: 7% (1/14) of DZP rats, 27% (3/11) of TC30 rats, 0% (0/10) of TC60 rats, 0% (0/11) of TC90 rats and 0% (0/9) of TC120 rats died. In the group of DZP rats, one rat died during the first 24 h after SE. In the group of TC30, two rats were dead by 24 h and one by 48 h after SE. Cell densities in hippocampus and cortex in the early phase (14 days after SE)

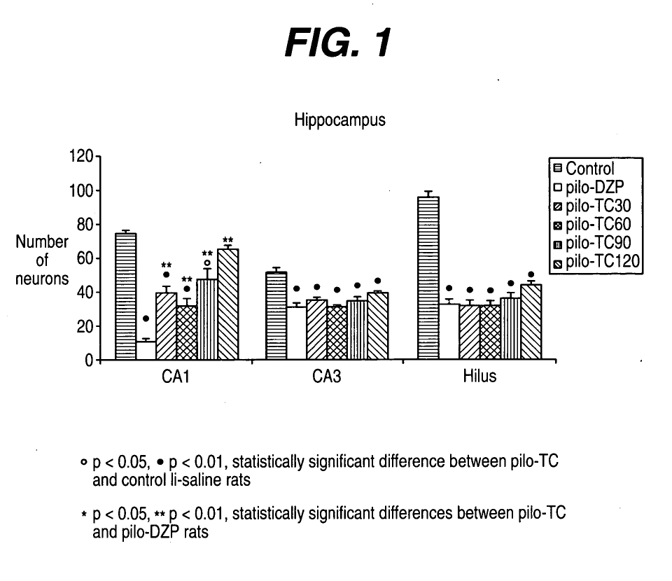

In DZP rats compared to control rats, the number of neurons was massively decreased in the CA1 region of the hippocampus (85% drop out in the pyramidal cell layer) while the CA3 region was less extensively damaged (40% loss) (Table 1 and FIG. 1). In the dentate gyrus, DZP rats experienced extensive neuronal loss in the hilus (65%) while the granule cell layer did not show overt damage. The same distribution of damage was observed in the ventral hippocampus but cell counts were not performed in this region.

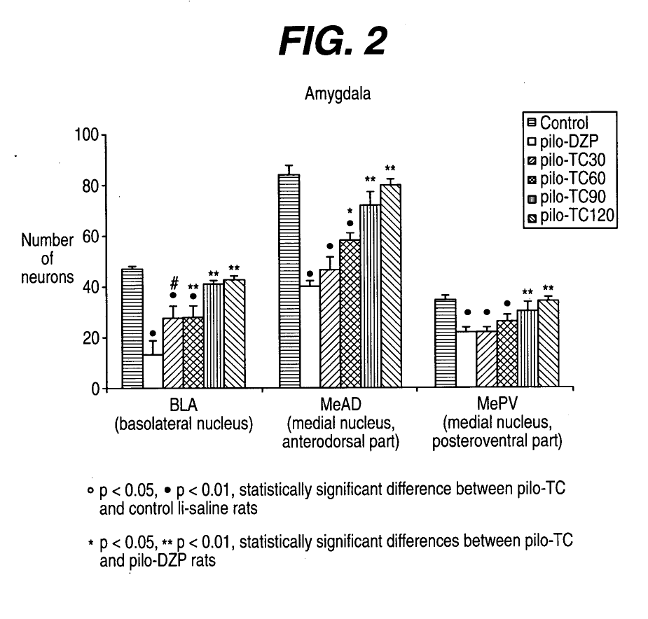

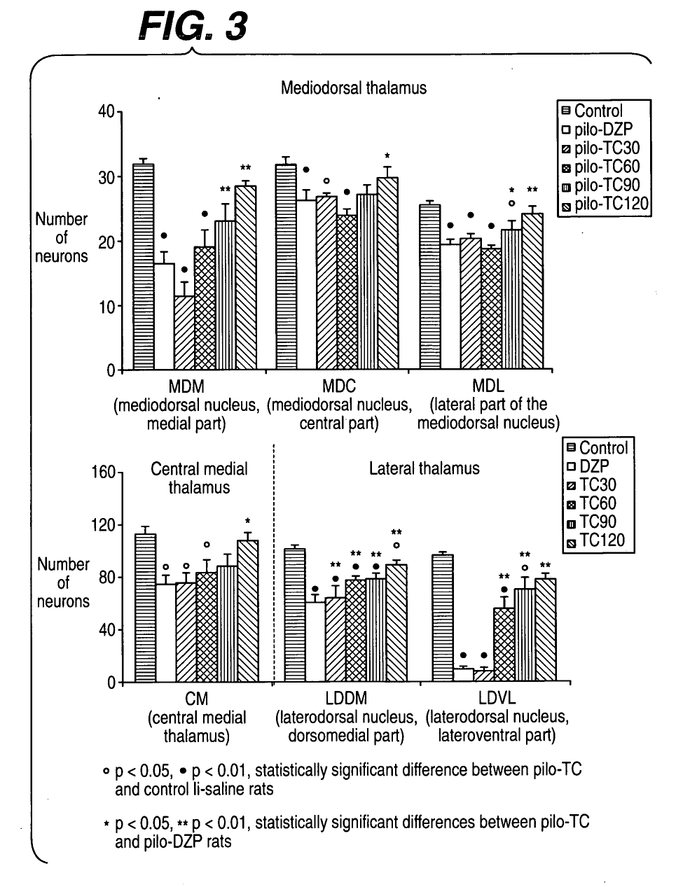

In the thalamus, neuronal loss was moderate in the mediodorsal central and lateral, the dorsolateral medial dorsal and in the central medial nuclei (18, 24, 40 and 34% drop out, respectively), more marked in the mediodorsal nucleus (49%) and major in the ventral lateral division of the dorsolateral nucleus (90%) (Table 1 and FIG. 2). In the amygdala, neuronal loss was moderate in the medial ventral posterior nucleus (38%) and more marked in the basolateral and medial dorsal anterior nuclei (73 and 53% drop out, respectively). There was no neuronal damage in the central nucleus (Table 1 and FIG. 3).

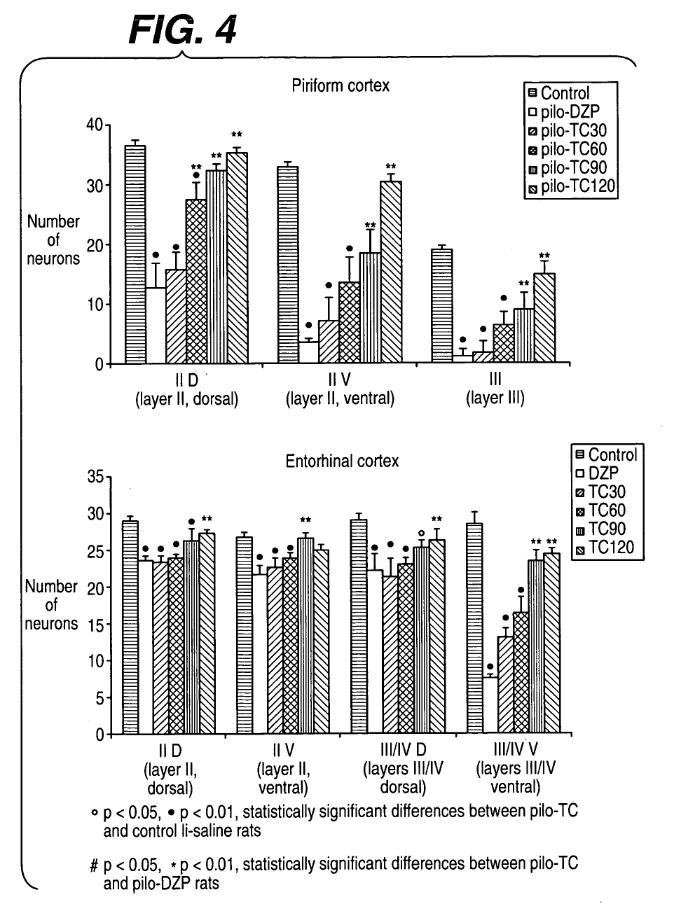

In the piriform cortex, neuronal loss was almost total in layer III (94%) which was no longer really visible and reached 66 and 89% in dorsal and ventral layer II, respectively in DZP rats compared to control saline-treated rats. In the dorsal entorhinal cortex, layers II and III-IV underwent slight damage (18 and 24%, respectively) and in ventral layers II and III/IV, damage reached 22 and 74%, respectively (Table 1 below and FIG. 4).

TABLE 1
Effects of increasing doses of TC on the number of neuronal cell bodies in the hippocampus,
thalamus, amygdala and cerebral cortex of rats subjected to li-pilo SE.

| | Control | pilo-DZP | pilo-TC30 | pilo-TC60 | pilo-TC90 | pilo-TC120 |
|---|---|---|---|---|---|---|
| | (n = 10) | (n = 7) | (n = 8) | (n = 11) | (n = 10) | (n = 8) |
| Hippocampus | | | | | | |
| CA1 area | 74.8 ± 1.5 | 10.9 ± 1.9** | 39.3 ± 4.4**°° | 31.9 ± 4.4**°° | 47.7 ± 6.6*° | 65.5 ± 2.9°° |
| CA3 area | 52.1 ± 2.7 | 31.3 ± 2.9** | 35.7 ± 1.8** | 31.6 ± 1.4** | 35.1 ± 2.9** | 39.8 ± 1.5** |
| Hilus | 96.4 ± 3.5 | 33.5 ± 3.0** | 33.0 ± 3.2** | 32.8 ± 3.3** | 37.5 ± 3.1** | 44.8 ± 2.9** |
| Thalamus | | | | | | |
| Mediodorsal | 31.9 ± 0.9 | 16.4 ± 1.9** | 11.5 ± 2.5** | 19.1 ± 2.6** | 23.1 ± 2.8°° | 28.6 ± 0.8°° |
| medial | | | | | | |
| Mediodorsal | 31.9 ± 1.2 | 26.3 ± 1.8** | 26.9 ± 0.6* | 24.1 ± 1** | 27.4 ± 1.5 | 29.9 ± 1.7° |
| central | | | | | | |
| Mediodorsal | 25.9 ± 0.6 | 19.6 ± 0.8** | 20.5 ± 0.7** | 18.9 ± 0.6** | 22 ± 1.2*° | 24.4 ± 1.1°° |
| lateral | | | | | | |
| Dorsolateral, | 102.2 ± 2.5 | 61 ± 6.3** | 64.2 ± 9.3**°° | 77.5 ± 3.9**°° | 79.4 ± 3.1**°° | 89.8 ± 3.7*° |
| medial, dorsal | | | | | | |
| Dorsolateral, | 97.8 ± 1.7 | 9.7 ± 2.5** | 8.8 ± 2.8** | 56.7 ± 8.7** | 71.8 ± 5.3°°* | 79.0 ± 4.7°° |
| ventral lateral | | | | | | |
| Central medial | 113.1 ± 5.9 | 74.2 ± 7.4* | 75.6 ± 7.7* | 83.7 ± 9.6* | 88.2 ± 8.5 | 108.2 ± 6.6° |
| Amygdala | | | | | | |
| Basolateral | 46.7 ± 1.2 | 12.8 ± 5.3** | 27.3 ± 4.9**° | 27.8 ± 4.3**°° | 40.7 ± 1.6°° | 42.7 ± 1.3°° |
| Medial, dorsal | 84.3 ± 3.8 | 40.0 ± 2.5** | 46.8 ± 5.0** | 58.4 ± 2.8**° | 72.2 ± 5.7°° | 80.2 ± 2.6°° |
| anterior | | | | | | |
| Medial, ventral | 35.1 ± 1.7 | 21.8 ± 2.4** | 22.3 ± 1.8** | 26.2 ± 2.9** | 30.7 ± 3.7°° | 34.7 ± 1.7°° |
| posterior | | | | | | |
| Cerebral | | | | | | |
| cortex | | | | | | |
| Piriform, layer | 36.6 ± 0.8 | 12.6 ± 4.2** | 15.7 ± 2.9** | 27.5 ± 2.8**°° | 32.4 ± 1.1°° | 35.2 ± 1.1°° |
| II, dorsal | | | | | | |
| Piriform, layer | 33.0 ± 0.8 | 3.6 ± 0.7** | 7.2 ± 3.8** | 13.7 ± 4.2** | 18.4 ± 4.0°° | 30.5 ± 1.3°° |
| II, ventral | | | | | | |
| Piriform, layer | 19.2 ± 0.7 | 1.2 ± 1.2** | 1.8 ± 1.8** | 6.4 ± 2.3** | 9 ± 3.0°° | 15 ± 2.2°° |
| III | | | | | | |
| Entorhinal, layer | 29 ± 0.6 | 23.5 ± 0.7** | 23.4 ± 0.6** | 23.9 ± 0.5** | 26.3 ± 0.9** | 27.3 ± 0.5°° |
| II, dorsal | | | | | | |
| Entorhinal, layer | 26.8 ± 0.7 | 21.7 ± 1.3** | 22.7 ± 0.9 | 23.3 ± 0.8** | 25.4 ± 1.1° | 25.1 ± 0.6 |
| II, ventral | | | | | | |
| Entorhinal, layer | 29.2 ± 0.9 | 22.3 ± 0.5** | 22.3 ± 0.5** | 23.2 ± 0.8** | 26.7 ± 0.8* | 26.4 ± 0.7°° |
| III/IV, dorsal | | | | | | |
| Entorhinal, layer | 28.7 ± 1.7 | 7.7 ± 2.3** | 13.2 ± 1.9** | 16.5 ± 2.2** | 23.7 ± 1.5°° | 24.5 ± 1.4°° |
| III/IV, ventral | | | | | | |

*p < 0.05,
**p < 0.01, statistically significant difference between pilo-TC and control li-saline rats
°p < 0.05,
°°p < 0.01, statistically significant differences between pilo-TC and pilo-DZP rats

In the hippocampus of TC-treated animals, cell loss was significantly reduced compared to DZP rats in CA1 pyramidal cell layer. This reduction was marked in TC30 , 60 or 90 rats (36-47% cell loss) and prominent in the TC120 group (12% cell loss). The differences were statistically significant at all TC doses (Table 1 and FIG. 1). In the CA3 pyramidal layer, there was a tendency to a slight neuroprotection induced by RWJ, only at the 120 mg/kg dose but the difference with the DZP group was not significant. In the dentate gyrus, the cell loss in the hilus was similar in the DZP and TC30 , 60 and 90 groups (61-66% drop out) and there was a slight tendency to reduced damage in the TC120 group (53% neuronal loss) compared to DZP animals (66% drop out). None of these differences was statistically significant.

In the thalamus, neuronal loss was similar in DZP and TC30 and TC60 rats. TC was significantly protective at the 60 mg/kg dose in the dorsolateral medial dorsal nucleus and at the two highest doses, 90 and 120 mg/kg in all thalamic nuclei, although the difference did not reach significance in the mediodorsal central and central medial nuclei in TC90 rats. In TC120 rats, neuronal drop out was considerably reduced compared to DZP rats. It ranged from 4-19% and the number of neurons was no longer significantly different from control animals, except in the dorsolateral medial dorsal nucleus (Table 1 and FIG. 2). In the amygdala, TC was significantly protective at the 30 mg/kg dose in the basolateral nucleus and at the 60 mg dose, also in the medial dorsal anterior nucleus. At the highest dose, TC was largely neuroprotective; the number of neurons was no longer significantly different from the control level and reached 86-99% of the control level in all amygdala nuclei (Table 1 and FIG. 3).

In the cerebral cortex, the treatment with TC did not significantly protect any cortical area compared to the DZP treatment at the dose of 30 mg/kg. At 60 mg/kg, TC significantly reduced neuronal loss only in layer II of the dorsal piriform cortex (25% drop out compared to 66% in the DZP group). At 90 and 120 mg/kg, TC significantly protected all three areas of the piriform cortex compared to the DZP treatment and at the highest dose of TC, 120 mg/kg, neuronal density reached 78-96% of control levels, even in piriform cortex, dorsal layer II and layer III where the neuronal population was almost totally depleted in the DZP group. In all layers of the dorsal and ventral entorhinal cortex, the two lowest doses of TC, 30 and 60 mg/kg did not afford any neuroprotection. The 90-mg/kg dose of TC significantly protected layers II and III/IV of the ventral entorhinal cortex (4 and 17% damage remaining in layers II and II/IV of the dorsal part and in layer II of the ventral part compared to 19 and 73% in the DZP group). At the highest dose of TC, 120 mg/kg, all parts of the entorhinal cortex, both dorsal and ventral were protected and the number of neurons in these areas was no longer significantly different from the level in controls (85-94% of neurons surviving compared to 27-81% in the DZP group).

Latency to and Frequency of Recurrent Seizures

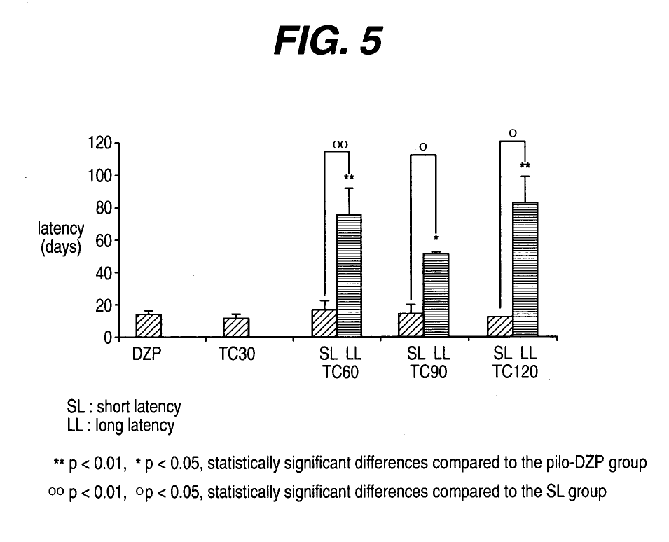

The latency to spontaneous seizures reached a mean value of 15.5±2.3 days in the DZP group (14 rats) and was similar (11.6±2.5 days) in the TC30 group (8 rats). At higher concentrations of TC, animals could be subdivided in subgroups with short and long latencies. A short latency was considered as any duration shorter than 40 days after SE. Some rats exhibited a latency to the first spontaneous seizure that was similar to that recorded in the DZP and TC groups but the number of rats exhibiting this short latency values progressively decreased with the increase in TC concentration. Thus at 30 mg/kg, 70% of the rats (7/10) had short latencies to seizures while at 90 and 120 mg/kg, this percentage reached 36% (4/11) and 11% (1/9), respectively (Table 2 below and FIG. 5).

TABLE 2
Effect of increasing doses of TC on the latency to spontaneous seizures.

| | Number of | Latency to the first spontaneous seizure | |
|---|---|---|---|
| Treatment | animals | (days) | |
| DZP | 14 | 15.5 ± 2.34 | |
| pilo-TC30 | 8 | 11.6 ± 2.5 | |
| | | 2 groups | |
| pilo-TC60 | 10 | Short latency (n = 7) | Long latency (n = 3) |
| | | 17.4 ± 5.4 | 76.7 ± 15.6** °° |

| | | 3 groups | | |
|---|---|---|---|---|
| pilo-TC90 | 11 | Short latency (n = 4) | Long latency (n = 2) | Non epileptic (n = 5) |
| | | 14.8 ± 5.7 | 52.0 ± 1.0* ° | 150** °° |
| | | 3 groups | | |
| pilo-TC120 | 9 | Short latency (n = 1) | Long latency (n = 4) | Non epileptic (n = 4) |
| | | 13.0 | 84.5 ± 16.7** °° | 150** °° |

**p < 0.01,
*p < 0.05, statistically significant differences compared to the pilo-DZP group
°° p < 0.01,
° p < 0.05, statistically significant differences compared to the short latency group

In the TC60, 90 and 120 groups, the mean value of the rats with long latencies was similar and ranged from 52 to 85 days. Finally, at the two highest doses of TC, we were able to identify a percentage of rats that did not develop any seizure over a duration of 150 days post-SE. The percentage of non-epileptic rats reached 45% at both doses of TC.

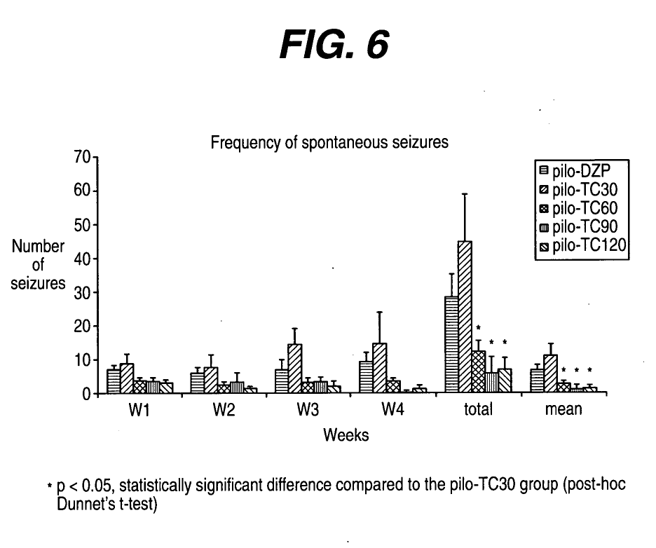

The frequency of spontaneous seizures was similar over the four weeks of recording. It showed a tendency to be higher in the DZP and TC30 groups while it was lower in the TC60, 90 and 120 groups (FIG. 6). These differences did not reach statistical significance at the level of each individual weekly frequency but reached significance for the total or mean number of seizures over the four weeks.

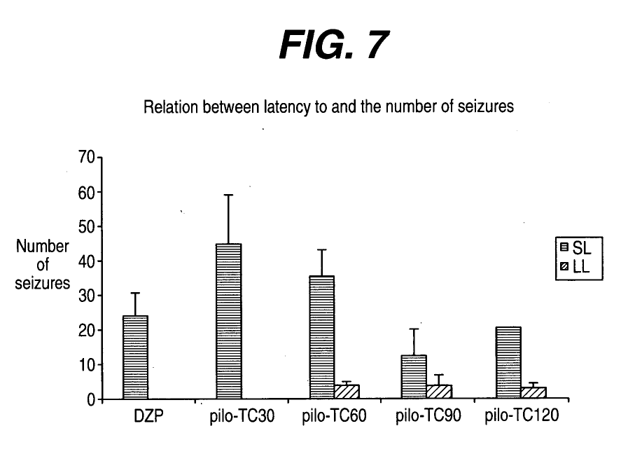

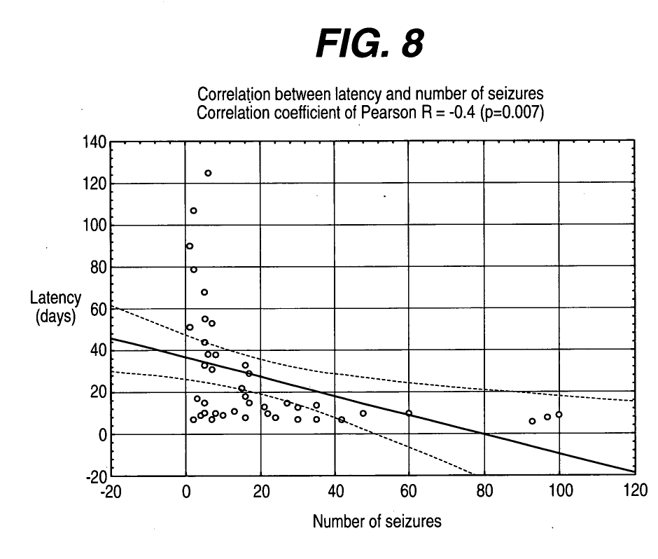

The number of seizures was also plotted according to the duration of the latency to the first spontaneous seizure. Animals with a short latency showed a tendency to display 2-3 times more seizures over the four weeks of recording than rats with a long latency period. No statistical analysis could be performed since the ANOVA did not show any significance, most likely because there was only one animal in the short.latency subgroup of the TC120 animals (FIG. 7). However, when all latency values were plotted against the number of seizures, there was a significant inverse correlation leading to a straight line with a correlation coefficient of −0.4 (FIG. 8).

To finalize this analysis, two more measurements will be performed. The first one is cell counting on the animals that were video recorded and followed for 2 months after the first spontaneous seizure or sacrificed at 5 months to study the potential correlation between the extent and location of brain damage and the occurrence of and/or latency to spontaneous seizures. The second one will be to perform a one-year follow-up of seizure occurrence in a group of rats to study whether or not the animals that we declare “non epileptic” at 5 months will remain seizure free.

The results of the present study show that a treatment with TC starting at 1 h after the onset of Li-pilo-induced SE has neuroprotective properties in the CA1 pyramidal cell layer of the hippocampus, and in all layers of the ventral and dorsal piriform and entorhinal cortex. TC protects also thalamus and amygdala nuclei. However, TC is not protective at the dose of 30 mg/kg, except in CA1, one thalamic and one amygdala nucleus. At the dose of 60 mg/kg, layer II of the dorsal piriform cortex and a second amygdala nucleus are also protected. At 90 and 120 mg/kg, the drug protects most cerebral regions studied, except hippocampal CA3 and the hilus of the dentate gyrus. The latter two structures plus the dorsolateral ventral dorsal thalamic nucleus are the only regions where the number of neurons remains significantly different from controls at the dose of 120 mg/kg TC. From these data, the extremely powerful neuroprotection properties of TC appear clearly. The molecule seems to prevent neuronal death in most regions belonging to the circuit of limbic epilepsy induced by Li-pilo, i.e., the hippocampus, thalamus, amygdale and parahippocampal cortices. These are all the regions in which we have detected MRI signal in the course of epileptogenesis in Li-pilo-treated rats (Roch et al., 2002a). The only two regions that are not efficiently protected by TC are CA3 pyramidal cell layer and the hilus of the dentate gyrus. The latter region undergoes rapid and massive cell damage (André et al., 2001; Roch et al., 2002a) and none of the neuroprotection that we used in previous studies have been able to protect this structure. We have also on the basis of earlier studies identified this structure as a key area in the initiation and maintenance of epileptic seizures in the Li-pilo model (Dubé et al., 2000). Obviously, the present data demonstrate that epileptogenesis can be prevented even though damage remains quite marked in this area. Long-term cell counting on the group of animals that has been video recorded will be able to show whether or not the extent of damage in this region is critical for epileptogenesis in this model.

The treatment did not affect the latency to the first spontaneous seizure at the dose of 30 mg/kg. At the 3 higher doses, a percentage of animals developed epilepsy as fast as the DZP or TC30 rats but the relative importance of this subgroup was inversely related to the dose of TC used. Another subgroup, constant in size (2-4 animals per group) developed epilepsy after a 4-6 times longer latency while at the two highest doses of the drug, 4-5 rats had not become epileptic after 5 months, i.e. about 10 times the duration of the short latency and 2-3 times that of the long latency. This delay in the occurrence of epilepsy might correlate with the number of neurons protected in the basal cortices in the animals. This assumption is based on the fact that we noted some heterogeneity in the extent of neuroprotection in basal cortices of the animals subjected the short term neuronal counting at 14 days after SE. However, at the moment, we have not performed neuronal counting in the animals used for the study of epileptogenesis and therefore, no conclusion can be drawn on a potential relation between the number of neurons surviving in basal cortices and the rate or even occurrence of epileptogenesis.

The data obtained in the present study are in line with the previous study from this group reporting that the 60-mg/kg dose of TC protected the hippocampus and the basal cortices from neuronal damage and delayed the occurrence of recurrent seizures (see previous report, 2002). They confirm that the protection of the basal cortices could be a key factor in inducing a disease modifying effect in the lithium-pilocarpine model of epilepsy. The key role of the basal cortices as initiators of the epileptic process was previously demonstrated by our group in the lithium-pilocarpine model (André et al., 2003; Roch et al., 2002a,b).

In conclusion, this study shows that the test compound (TC) has very promising anti-epileptogenic effects.

### References for Example 2

André V, Marescaux C, Nehlig A, Fritschy J M (2001) Alterations of the hippocampal GABAergic system contribute to the development of spontaneous recurrent seizures in the lithium-pilocarpine model of temporal lobe epilepsy. Hippocampus 11:452-468.
André V, Rigoulot M A, Koning E, Ferrandon A, Nehlig A (2003) Long-term pregabalin treatment protects basal cortices and delays the occurrence of spontaneous seizures in the lithium-pilocarpine model in the rat. Epilepsia 44:893-903.
Cavalheiro E A (1995) The pilocarpine model of epilepsy. Ital J Neurol Sci 16:33-37.
Dubé C, Marescaux C, Nehlig A (2000) A metabolic and neuropathological approach to the understanding of plastic changes occurring in the immature and adult rat brain during lithium-pilocarpine induced epileptogenesis. Epilepsia 41 (Suppl 6):S36-S43.
Dubé C, Boyet S, Marescaux C, Nehlig A (2001) Relationship between neuronal loss and interictal glucose metabolism during the chronic phase of the lithium-pilocarpine model of epilepsy in the immature and adult rat. Exp Neurol 167:227-241.
Paxinos G, Watson C (1986) The Rat Brain in Stereotaxic Coordinates, 2nd ed. Academic Press, San Diego.
Roch C, Leroy C, Nehlig A, Namer I J (2002a) Contribution of magnetic resonance imaging to the study of the lithium-pilocarpine model of temporal lobe epilepsy in adult rats. Epilepsia 43:325-335.
Roch C, Leroy C, Nehlig A, Namer I J (2002b) Predictive value of cortical injury for the development of temporal lobe epilepsy in P21-day-old rats: a MRI approach using the lithium-pilocarpine model. Epilepsia 43:1129-1136.
Turski L, Ikonomidou C, Turski W A, Bortolotto Z A, Cavalheiro E A (1989) Review: Cholinergic mechanisms and epileptogenesis. The seizures induced by pilocarpine: a novel experimental model of intractable epilepsy. Synapse 3:154-171.
   References Cited

All references cited herein are incorporated herein by reference in their entirety and for all purposes to the same extent as if each individual publication or patent or patent application was specifically and individually indicated to be incorporated by reference in its entirety for all purposes.

The discussion of references herein is intended merely to summarize the assertions made by their authors and no admission is made that any reference constitutes prior art. Applicants reserve the right to challenge the accuracy and pertinence of the cited references.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

What is claimed is:

1. A method for treating epileptogenesis, comprising administering to a patient in need of treatment with an anti-epileptogenic drug (an AEGD) a therapeutically effective amount of a compound, or a pharmaceutically acceptable salt or ester thereof, selected from the group consisting of Formula (I) and Formula (II): 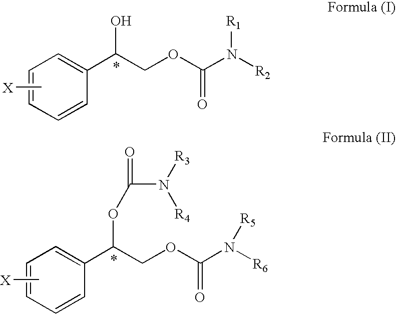
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

2. The method of claim 1 wherein X is chlorine.

3. The method of claim 1 wherein X is substituted at the ortho position of the phenyl ring.

4. The method of claim 1 wherein R₁, R₂, R₃, R₄, R₅ and R₆ are selected from hydrogen.

5. A method for treating epileptogenesis, comprising administering to a patient in need of treatment with an anti-epileptogenic drug (an AEGD) a therapeutically effective amount of an enantiomer, or a pharmaceutically acceptable salt or ester thereof, selected from the group consisting of Formula (I) and Formula (II) or enantiomeric mixture wherein one enantiomer selected from the group consisting of Formula (I) and Formula (II) predominates: 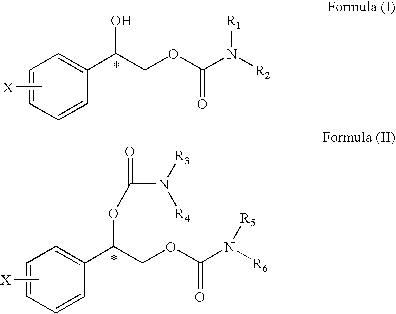
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

6. The method of claim 5 wherein X is chlorine.

7. The method of claim 5 wherein X is substituted at the ortho position of the phenyl ring.

8. The method of claim 5 wherein R₁, R₂, R₃, R₄, R₅ and R₆ are selected from hydrogen.

9. The method of claim 5 wherein one enantiomer selected from the group consisting of Formula (I) and Formula (II) predominates to the extent of about 90% or greater.

10. The method of claim 5 wherein one enantiomer selected from the group consisting of Formula (I) and Formula (II) predominates to the extent of about 98% or greater.

11. The method of claim 5 wherein the enantiomer selected from the group consisting of Formula (I) and Formula (II) is an enantiomer selected from the group consisting of Formula (Ia) and Formula (IIa): 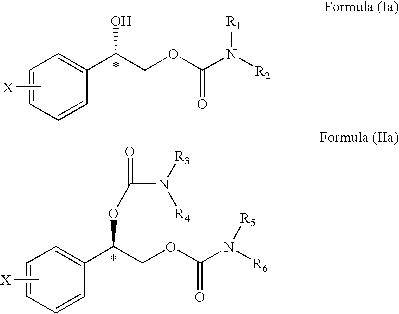
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

12. The method of claim 11 wherein X is chlorine.

13. The method of claim 11 wherein X is substituted at the ortho position of the phenyl ring.

14. The method of claim 11 wherein R₁, R₂, R₃, R₄, R₅ and R₆ are selected from hydrogen.

15. The method of claim 11 wherein one enantiomer selected from the group consisting of Formula (Ia) and Formula (IIa) predominates to the extent of about 90% or greater.

16. The method of claim 11 wherein one enantiomer selected from the group consisting of Formula (Ia) and Formula (IIa) predominates to the extent of about 98% or greater.

17. The method of claim 5 wherein the enantiomer selected from the group consisting of Formula (I) and Formula (II) is an enantiomer selected from the group consisting of Formula (Ib) and Formula (IIb) or a pharmaceutically acceptable salt or ester form thereof:

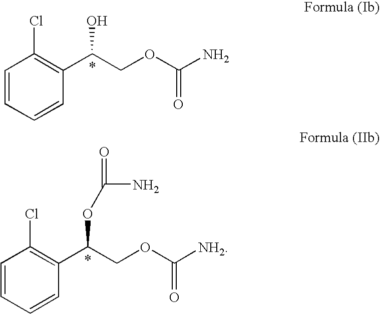

18. The method of claim 17 wherein one enantiomer selected from the group consisting of Formula (Ib) and Formula (IIb) predominates to the extent of about 90% or greater.

19. The method of claim 17 wherein one enantiomer selected from the group consisting of Formula (Ib) and Formula (IIb) predominates to the extent of about 98% or greater.

20. The method, as claimed in claims 1 or 5 wherein the predisposing factor(s) rendering the patient in need of treatment with an anti-epileptogenic drug (an AEGD) are selected from the group consisting of: injury or trauma of any kind to the CNS; infections of the CNS; anoxia; stroke (CVAs); autoimmune diseases affecting the CNS, e.g., lupus; birth injures, e.g., perinatal asphyxia; cardiac arrest; therapeutic or diagnostic vascular surgical procedures, e.g., carotid endarterectomy or cerebral angiography; spinal cord trauma; hypotension; injury to the CNS from emboli, hyper or hypo perfusion; hypoxia; known genetic predisposition to disorders known to respond to AEGDs; space occupying lesions of the CNS; brain tumors, e.g., glioblastomas; bleeding or hemorrhage in or surrounding the CNS, e.g., intracerebral bleeds or subdural hematomas; brain edema; febrile convulsions; hyperthermia; exposure to toxic or poisonous agents; drug intoxication or withdrawal, e.g. cocaine or alcohol; family history of; seizure disorders or an epilepsy related seizure like neurological phenomenon or seizure related disorder, history of status epilepticus; current treatment with medications that lower seizure threshold, e.g., lithium carbonate, thorazine or clozapine; evidence from surrogate markers or biomarkers that the patient is in need of treatment with an anti-epileptogenic drug, e.g. MRI scan showing hippocampal sclerosis, elevated serum levels of neuronal degradation products, elevated levels of ciliary neurotrophic factor (CNTF) or an EEG suggestive of a seizure disorder or an epilepsy related seizure like neurological phenomenon or an analogous seizure related disorder.

21. The method of claim 20 wherein the predisposing factor(s) rendering the patient in need of treatment with an anti-epileptogenic drug (an AEGD) are selected from the group consisting of: closed or penetrating head trauma; stroke or other cerebral-vascular accident (CVA); status epilepticus and space occupying lesions of the CNS.

22. The method of claim 21 wherein the said predisposing factor(s) are closed or penetrating head trauma.

23. The method of claim 21 wherein the said predisposing factor(s) are stroke or other cerebral-vascular accident (CVA).

24. The method of claim 23 wherein the said predisposing factor is status epilepticus.

25. The methods of claims 1 or 5 wherein said compound (or enantiomer) or a pharmaceutically acceptable salt or ester thereof is administered in combination administration with one or more other compounds or therapeutic agents.

26. The methods of claim 25 wherein the said one or more other compounds or therapeutic agents are selected from the group consisting of compounds that have one or more of the following properties: antioxidant activity; NMDA receptor antagonism; ability to augment endogenous GABA inhibition; NO synthase inhibitor activity; iron binding ability, e.g., an iron chelator; calcium binding ability, e.g., a Ca (II) chelator; zinc binding ability, e.g., a Zn (II) chelator; the ability to block sodium or calcium ion channels; the ability to open potassium or chloride ion channels; such that epileptogenesis is inhibited in the patient.

27. The methods of claim 26 wherein the said one or more compounds may, in addition, be selected from the group consisting of anti-epileptic drugs (AEDs).

28. The methods of claim 27 wherein the said anti-epileptic drug (AED) is selected from the group consisting of; carbamazepine, clobazam, clonazepam, ethosuximide, felbamate, gabapentin, lamotigine, levetiracetam, oxcarbazepine, phenobarbital, phenytoin, pregabalin, primidone, retigabine, talampanel, tiagabine, topiramate, valproate, vigabatrin, zonisamide, benzodiazepines, barbiturates or sedative hypnotics.

29. A pharmaceutical composition for treating epileptogenesis comprising a pharmaceutically effective amount of an enantiomer, or a pharmaceutically acceptable salt or ester thereof, selected from the group consisting of Formula (I) and Formula (II) or enantiomeric mixture wherein one enantiomer selected from the group consisting of Formula (I) and Formula (II) predominates: 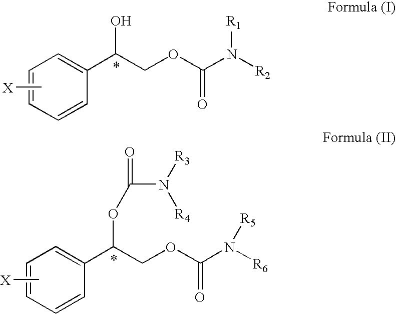
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano) and a pharmaceutically acceptable carrier or excipient.

30. A kit, comprising therapeutically effective dosage forms of the pharmaceutical composition claimed in claim 29 in an appropriate package or container together with information or instructions for proper use thereof.

31. The method as in claims 1 or 5 wherein the therapeutically effective amount is from about 0.01 mg/Kg/dose to about 100 mg/Kg/dose.

32. The method, as claimed in claims 1 or 5, wherein said patient has not developed epilepsy at the time of said administration.

33. The method, as claimed in claims 1 or 5, wherein said patient is at risk for developing epilepsy at the time of said administration.

34. The method, as claimed in claims 1 or 5, wherein said patient has developed epilepsy at the time of said administration.

35. The method, as claimed in claims 1 or 5, wherein the said therapeutic amount is progressively decreased as the treatment of the epileptogenic process progresses in the said patient.

36. The method, as claimed in claims 25, 26, 27 or 28 wherein the amount of the said one or more other compounds or therapeutic agents administered in combination with the said compound (or enantiomer) or a pharmaceutically acceptable salt or ester thereof is progressively decreased as the treatment of the epileptogenic process progresses in the said patient.
